(19) United States
(12) Patent Application Publication
(10) Pub. No.: US 2026/0248625 A1
(43) Pub. Date: Aug. 27, 2026

(54) INTEGRATED MULTIMODAL SENSORY AND INTELLIGENT ADAPTIVE CONTROL SYSTEM AND METHOD FOR PROSTHETIC LIMBS

(71) Applicant: KING FAHD UNIVERSITY OF PETROLEUM AND MINERALS, Dhahran (SA)

(72) Inventor: Ammar Ayad ALZAYDI, Dhahran (SA)

(73) Assignee: KING FAHD UNIVERSITY OF PETROLEUM AND MINERALS, Dhahran (SA)

(21) Appl. No.: 19/064,126

(22) Filed: Feb. 26, 2025

(51) Int. Cl. A61F 2/64; A61B 5/389; A61F 2/66; A61F 2/72

(52) U.S. Cl. CPC A61F 2/64; A61B 5/389; A61F 2/6607; A61F 2/72

(57) ABSTRACT

A prosthetic limb for replacing a portion of a leg of a subject includes a prosthetic limb body, a prosthetic limb knee, a prosthetic limb ankle and a multimodal sensor array to obtain sensor data used to derive parameters indicative of a physiological state of the subject and an environment in a proximity of the prosthetic limb. The prosthetic limb includes a data processing unit to process the sensor data to generate a processed sensor data generated using a machine learning (ML) model. The ML model outputs the processed sensor data as a predicted subject intent and predicted prosthetic limb parameters. The prosthetic limb includes a control system to generate a control signal having control parameters based on the processed sensor data using a fuzzy logic controller and a proportional-integral-derivative (PID) controller of the control system for adjusting a movement of the prosthetic limb.

### BACKGROUND

### Technical Field

The present disclosure is directed to prosthetics and, more particularly, to an integrated multimodal sensory and intelligent adaptive control system and method for prosthetic limbs.

### Description of Related Art

The “background” description provided herein is for the purpose of generally presenting the context of the disclosure. Work of the presently named inventors, to the extent it is described in this background section, as well as aspects of the description which may not otherwise qualify as prior art at the time of filing, are neither expressly or impliedly admitted as prior art against the present invention.

The field of prosthetic limbs has made significant progress over the past few decades, transitioning from basic mechanical devices to advanced systems capable of replicating natural human motion. Despite these advancements, many modern prosthetics still face significant limitations, particularly in their ability to adapt dynamically to user's changing environment and activity levels. Currently, prosthetic systems rely on static control mechanisms, which lack flexibility to accommodate variability in user movement patterns and environmental interactions on a daily basis.

Conventionally, prosthetic limb technology has focused on replicating basic functions of missing limbs through mechanical means or basic electronic and robotic systems. Early prosthetics were passive devices designed to provide minimal functional support and cosmetic resemblance without adaptive capabilities. With the advent of microprocessors and improved materials technology, the prosthetics evolved to include active components such as motors and actuators controlled by limited sensor inputs, typically from electromyographic (EMG) signals. These advances led to the development of myoelectric prosthetics, which use electrical activity from residual muscles to control the movements of the prosthetic limbs. While this was a significant improvement, these systems still rely heavily on predefined patterns of movement that do not adjust in real-time to changing external conditions or user's specific activity levels. Furthermore, such systems often require significant user effort to master and can be unintuitive, as control mechanisms do not directly correspond to natural feedback mechanisms of biological limbs.

Further, intelligent control systems have been introduced to address the aforementioned issues that enhance adaptability and responsiveness of the prosthetics. Such systems are designed to process a multitude of sensor inputs, including limb position, environmental conditions, and even neuromuscular signals from users. However, a full potential of integrating advanced sensing technologies and adpative algorithms into the prosthetic limbs has yet to be fully realized in a way that mimics a true complexity and fluidity of natural human movement.

Further, various other proesthetic systems have been developed. However, such prosthetic systems lack the ability to dynamically adjust to different environmental contexts or the user's varied physical activities. This can lead to impractical or inefficient use in everyday situations, such as navigating uneven terrain or altering walking speed. Moreover, these prosthetic systems often rely on a narrow range of sensors, primarily focused on detecting muscle activity through the EMG signals. This limits a type and an amount of data available for controlling the prosthetics, leading to a gap between user's intent and a response of the prosthetic limb. Additionally, these prosthetics may be cumbersome and unintuitive for the user, requiring significant effort to operate, which often results in a steep learning curve and user fatigue.

Thus, there is a need for an improved and advanced prosthetic limb that can overcome shortcomings of the existing prior arts.

### SUMMARY

In an exemplary embodiment, a prosthetic limb for replacing a portion of a leg of a subject is described. The prosthetic limb includes a prosthetic limb body, a prosthetic limb knee and a prosthetic limb ankle. The prosthetic limb further includes a multimodal sensor array embedded in the prosthetic limb body to obtain sensor data. The sensor data are used to derive parameters that are indicative of (a) a physiological state of the subject and (b) an environment in proximity of the prosthetic limb. The prosthetic limb further includes a data processing unit that is configured to process the sensor data to generate processed sensor data. The processed sensor data is generated using a machine learning (ML) model trained on training data including the sensor data, corresponding subject intent and corresponding prosthetic limb parameters. The ML model outputs the processed sensor data as a predicted subject intent and predicted prosthetic limb parameters based on the sensor data. The prosthetic limb further includes a control system that is configured to generate a control signal having control parameters for adjusting a movement of the prosthetic limb. The control parameters include values of the prosthetic limb parameters to be adjusted. The control system is configured to generate the control signal based on the processed sensor data using a fuzzy logic controller and a proportional-integral-derivative (PID) controller of the control system.

In another exemplary embodiment, a method for controlling a prosthetic limb is described. The method includes acquiring multimodal sensor data. The multimodal sensor data is collected from a plurality of sensors integrated into the prosthetic limb, including: accelerometers and gyroscopes for linear and angular motion, pressure sensors for force distribution across a foot, electromyography (EMG) sensors, and environmental sensors including inclinometers for slope detection and ultrasonic sensors for obstacle detection. The method further includes processing the sensor data. The processing includes filtering and normalizing the sensor data to remove noise and ensure consistency in data format. The processing further includes extracting features from the sensor data to identify subject movement patterns and environmental conditions. The processing further includes generating predicted prosthetic limb parameters and predicted subject intent. The features are input into a machine learning (ML) model trained on training data including sensor data, corresponding subject intent and corresponding prosthetic limb parameters. The ML model outputs the predicted subject intent and the predicted prosthetic limb parameters. The processing further includes generating a control signal based on the processed sensor data. The control signal is generated by a control system comprising a fuzzy logic controller and a proportional-integral-derivative (PID) controller. The control signal is used to adjust the prosthetic limb parameters in real time.

The foregoing general description of the illustrative embodiments and the following detailed description thereof are merely exemplary aspects of the teachings of this disclosure, and are not restrictive.

### BRIEF DESCRIPTION OF THE DRAWINGS

A more complete appreciation of this disclosure and many of the attendant advantages thereof will be readily obtained as the same becomes better understood by reference to the following detailed description when considered in connection with the accompanying drawings, wherein:

FIG. 1A illustrates an exemplary subject wearing a prosthetic limb, according to certain embodiments.

FIG. 1B illustrates non-invasive sensory attachments positioned on an unaffected leg, according to certain embodiments.

FIG. 1C illustrates a placement of accelerometers, according to certain embodiments.

FIG. 2A illustrates a block diagram of a prosthetic limb management system, according to certain embodiments.

FIG. 2B illustrates a schematic representation of functional components of the prosthetic limb, according to certain embodiments.

FIG. 3 illustrates a flowchart of a process representing a detailed visualization of controlling the prosthetic limb, according to certain embodiments.

FIG. 4 illustrates a flowchart of a method for adaptive control of the prosthetic limb using multi-sensor integration, according to certain embodiments.

FIG. 5 illustrates an exemplary flowchart of a method representing output adjustment mechanisms in the prosthetic limb, according to certain embodiments.

FIG. 6 illustrates a component interaction diagram for the prosthetic limb, according to certain embodiments.

FIG. 7 illustrates a treadmill-based prosthetic limb testing system, according to certain embodiments.

FIG. 8 illustrates a prosthetic limb testing system using an Adaptive Prosthetic Knee (APK) in a controlled environment, according to certain embodiments.

FIG. 9 illustrates a prosthetic limb prototype, according to certain embodiments.

FIG. 10 illustrates a visualization of prosthetic control adaptation, according to certain embodiments.

FIG. 11 illustrates a visual representation of simulated data collected over a predefined period, according to certain embodiments.

FIG. 12 illustrates an exemplary graph representing stability on varied terrain, according to certain embodiments.

FIG. 13 illustrates a hybrid fuzzy-proportional-integral-derivative (PID) control architecture, according to certain embodiments.

FIGS. 14A and 14B illustrate input membership functions of a fuzzy logic controller, according to certain embodiments.

FIG. 15 illustrates a rule-based fuzzy inference process used in the fuzzy logic controller, according to certain embodiments.

FIG. 16 illustrates a flowchart of a method for training a machine learning model, according to certain embodiments.

FIG. 17 illustrates a flowchart of a method for enabling slope navigation and obstacle detection in the prosthetic limb, according to certain embodiments.

FIG. 18 is an illustration of a non-limiting example of details of computing hardware used in a computing system, according to certain embodiments.

FIG. 19 is an exemplary schematic diagram of a data processing system used within the computing system, according to certain embodiments.

FIG. 20 is an exemplary schematic diagram of a processor used with the computing system, according to certain embodiments.

FIG. 21 is an illustration of a non-limiting example of distributed components that may share processing with a controller, according to certain embodiments.

### DETAILED DESCRIPTION

In the drawings, like reference numerals designate identical or corresponding parts throughout the several views. Further, as used herein, the words “a,” “an” and the like generally carry a meaning of “one or more,” unless stated otherwise.

Furthermore, the terms “approximately,” “approximate,” “about,” and similar terms generally refer to ranges that include the identified value within a margin of 20%, 10%, or preferably 5%, and any values therebetween.

Aspects of this disclosure are directed to a system and method for enhancing autonomy and functionality of prosthetic limbs through a combination of integrated multimodal sensory approach and adaptive control algorithms. Conventional prosthetic systems often lack the capability to dynamically adapt to varying terrain types and user activities, resulting in unnatural responses and limited functionality. Such systems rely on static control mechanisms that fail to account for complexities of real-world environments and user-specific movements, leading to suboptimal performance and user dissatisfaction.

The present disclosure relates to a system and method that leverages a combination of environmental sensors, user-input feedback mechanisms, and other motion sensors to gather detailed data about user's movements and surrounding conditions. Multimodal sensory data is processed in real-time using machine learning techniques, enabling the prosthetic limb to dynamically adapt its movements to align seamlessly with user's intent. Also, the system utilizes predictive analytics to anticipate user actions, facilitating smoother transitions and more natural responses across various activities and terrains. The system also provides output that includes precise motor control adjustments, real-time haptic feedback, and interactive visual and audio cues through a connected user interface. Further, the system advances the capabilities of prosthetic technologies by addressing limitations of existing systems, such as limited adaptability, unnatural motion mimicry, and poor user interactivity.

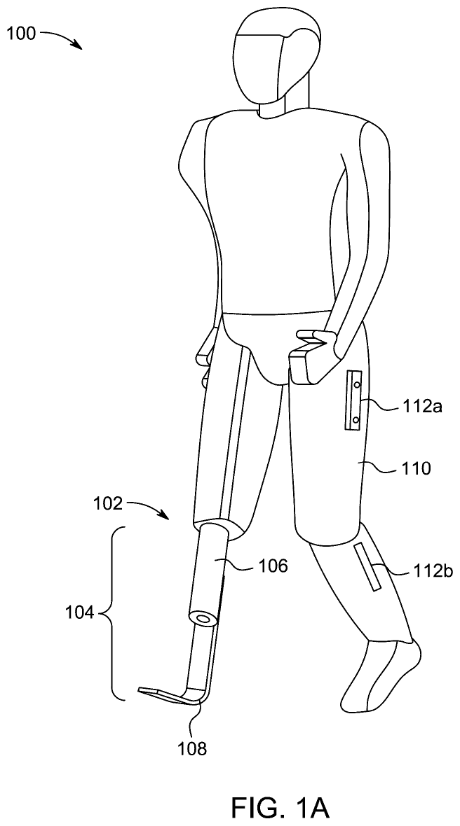

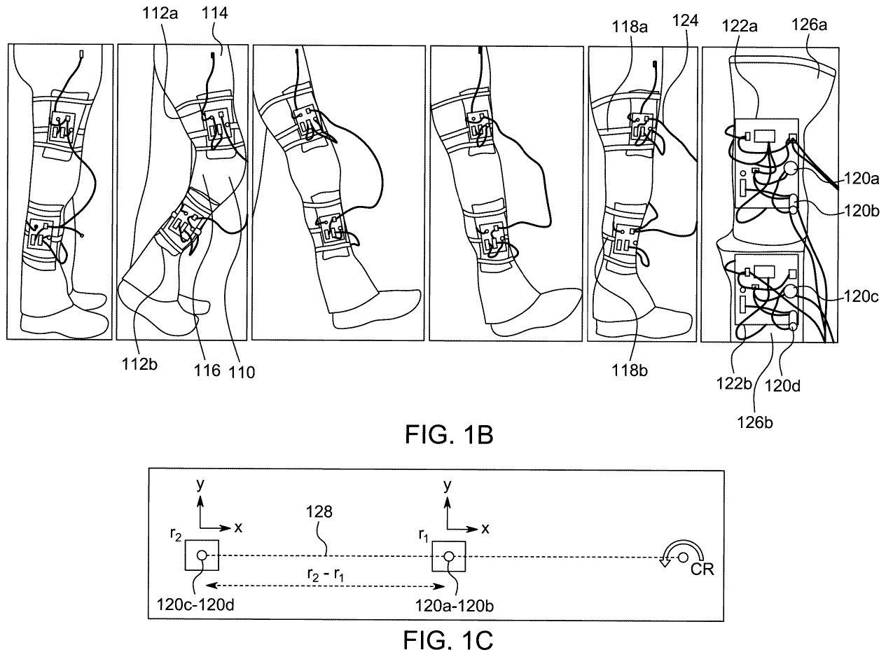

FIG. 1A illustrates an exemplary subject 100 wearing a prosthetic limb 102, according to certain embodiments. As used herein, the term “subject 100” refers to an individual participating in a study, experiment, or demonstration. In a preferred embodiment, the subject 100 is a human wearing the prosthetic limb 102. Also, as used herein, the term “prosthetic limb 102” refers to an artificial device designed to replace a missing limb (i.e., arm or leg) of the subject 100, enabling individuals with amputations to perform activities similar to those performed with natural limbs. In a preferred embodiment, the prosthetic limb 102 is a prosthetic leg designed to replace a portion of the leg of the subject 100. The prosthetic limb 102 includes components such as a prosthetic limb body 104, a prosthetic limb knee 106 and a prosthetic limb ankle 108.

In an embodiment, the prosthetic limb body 104 forms a structural framework of the prosthetic limb 102, providing stability and support to the prosthetic limb 102. The prosthetic limb 102 enables the subject 100 to walk, stand or perform various activities (e.g., climbing stairs and running) with ease. In an embodiment, the prosthetic limb body 104 may be made up of lightweight, durable, and high-strength materials to ensure stability, flexibility, and comfort while minimizing weight. The materials used for making the prosthetic limb body 104 may include, but are not limited to, titanium, carbon fiber, aluminium, thermoplastics, stainless steel, and the like. Embodiments of the present disclosure are intended to include or otherwise cover any suitable materials for the prosthetic limb body 104, including known related art and/or later developed materials.

The prosthetic limb knee 106 is essential for the individuals with above-the-knee amputations, allowing controlled flexion and leg extension for natural walking and movement. In an embodiment, the prosthetic limb knee 106 may incorporate various mechanisms to replicate a function of a natural knee. The various mechanisms may include, but are not limited to, a mechanical knee, a pneumatic knee, a microprocessor-controlled knee and so forth. Embodiments of the present disclosure are intended to include or otherwise cover any mechanism for the functioning of the prosthetic limb knee 106, including known related art and/or later developed technologies.

The prosthetic limb ankle 108 is designed for the individuals with below-the-knee amputations. The prosthetic limb ankle 108 enables natural foot movement and provides stability while standing, walking and running. In an embodiment, the prosthetic limb ankle 108 may incorporate various mechanisms to simulate human ankle motion. The mechanisms may be, but are not limited to, a solid ankle, a dynamic ankle, a microprocessor-controlled ankle, and so forth. Embodiments of the present disclosure are intended to include or otherwise cover any mechanism for the functioning of the prosthetic limb ankle 108, including known related art and/or later developed technologies. Each component of the prosthetic limb 102 works in coordination to restore mobility and functionality, enabling the individual, such as the subject 100 to perform daily activities with improved comfort and control.

To enhance effectiveness and validate the performance of the prosthetic limb 102, non-invasive sensory attachments 112a-112b (explained in detail in FIG. 1B) may be positioned on an unaffected leg 110 of the subject 100. As used herein, the “non-invasive sensory attachments 112a-112b” refer to external sensors or devices that may be attached to a body or the prosthetic limb 102 without penetrating the skin or requiring surgical implantation. The non-invasive sensory attachments 112a-112b collect biomechanical data, such as joint angles, gait patterns (i.e., walking patterns), and muscle activity from the unaffected leg 110 during the movement. The collected biomechanical data serves as a reference for calibrating and optimizing the prosthetic limb 102, enabling the prosthetic limb 102 to mimic natural movement patterns of the unaffected leg 110 accurately. In an embodiment, an integration of the prosthetic limb 102 with the non-invasive sensory attachments 112a-112b ensures seamless adaptation to the daily activities of the subject 100 while maintaining a natural and balanced gait.

FIG. 1B illustrates the non-invasive sensory attachments 112a-112b positioned on the unaffected leg 110, according to certain embodiments. In an embodiment, the non-invasive sensory attachments 112a-112b may be positioned on femur 114 and tibia 116 (i.e., above and below the knee) of the unaffected leg 110 using adjustable straps 118a-118b. The adjustable straps 118a-118b may ensure a secure and comfortable fit, preventing unwanted displacement during motion while accommodating different leg sizes and anatomies. The adjustable straps 118a-118b may include, but are not limited to, Velcro straps, elastic straps with buckles, nylon webbing straps with slide adjusters, silicon straps, and the like. Embodiments of the present disclosure are intended to include or otherwise cover any type of the adjustable straps 118a-118b, including known related art and/or later developed technologies.

The non-invasive sensory attachments 112a-112b may include at least two pairs of accelerometers 120a-120d (i.e., three-axis accelerometers) that may be positioned on each axis of the unaffected leg 110. In an embodiment, a first pair of accelerometers 120a-120b may be affixed on the femur 114 of the unaffected leg 110 and a second pair of accelerometers 120c-120d may be affixed on the tibia 116 of the unaffected leg 110.

In an embodiment, the pairs of accelerometers 120a-120d may be embedded within electronic boards 122a-122b. The electronic boards 122a-122b may also include microcontrollers, data transmission modules, and so forth. In an exemplary embodiment, the microcontrollers may be configured to process sensor data, and the data transmission modules may be configured to transmit the processed sensor data to a remote server for further analysis.

In an embodiment, the electronic boards 122a-122b may be connected to each other through wired connections 124, enabling seamless data transmission between the non-invasive sensory attachments 112a-112b. The wired connections 124 ensure synchronized data collection from the pairs of accelerometers 120a-120d embedded in the electronic boards 122a-122b, facilitating accurate measurement of the biomechanical data. In an embodiment, the electronic boards 122a-122b may be mounted on a layer of corresponding wraps (e.g., cloth) 126a-126b, acting as a protective barrier between the non-invasive sensory attachments 112a-112b and the unaffected leg 110. The protective barrier ensures the stability of the non-invasive sensory attachments 112a-112b on the unaffected leg 110, thus eliminating movement artifacts that may affect data accuracy.

In an exemplary embodiment, the non-invasive sensory attachments 112a-112b may continuously capture gait phase data by monitoring the movement of the unaffected leg 110. As used herein, the term “gait phase data” refers to information collected from the multiple sensors during different phases of walking (gait cycle). As the individual moves through different phases of the gait cycle, the accelerometers 120a-120d embedded in the electronic boards 122a-122b may track a real-time position of the femur 114, the tibia 116 and an ankle to collect the biomechanical data. Further, the collected biomechanical data is transmitted through the wired connections 124 between the electronic boards 122a-122b, ensuring synchronized measurements across different limb segments. Further, the biomechanical data is processed by the microcontrollers to analyze the gait dynamics (e.g., step frequency, stride length, and pace) and optimize response of the prosthetic limb 102.

As shown in FIG. 1B, the different phases of the gait cycle may include a first phase when the foot first makes contact with a ground, a second phase when an entire foot comes into contact with the ground, and weight shifts onto a stance leg (i.e., leg that is in contact with the ground and supporting the body's weight during walking or running), a third phase when the body moves forward over the stance leg, balancing on one foot while preparing for next foot, a fourth phase when a heel lifts off the ground as the body continues to shift forward, transferring weight to a front of the foot, a fifth phase when toes push off from the ground, initiating a swing phase as the leg begins to lift, a sixth phase when the leg moves forward, lifting off completely and clearing the ground, a seventh phase when the leg reaches its highest point in the air while continuing its forward motion and an eighth phase when the leg prepares to make contact with the ground again, completing the gait cycle.

FIG. 1C illustrates a placement of the accelerometers 120a-120d, according to certain embodiments. In an embodiment, the pairs of accelerometers 120a-120d may measure an angular acceleration and an angular velocity by capturing dynamic motion at specific points on the unaffected leg 110. As used herein, the term “angular acceleration” refers to a rate at which the angular velocity of an object changes with respect to time. Also, as used herein, the term “angular velocity” refers to a rate at which the object moves around a particular axis. In an embodiment, two accelerometers from different pairs (e.g., a first accelerometer 120a from the first pair and a second accelerometer 120c from the second pair) may be placed apart at a predefined distance D (denoted as 12-11) 128 that may be useful for accurate dynamic angle measurements. The accelerometers 120a and 120c may track the movement and the acceleration of the unaffected leg 110 along a specific axis (i.e., x direction and y direction). In an embodiment, a radial acceleration may be measured by the first accelerometer 120a and the second accelerometer 120c in the x-direction using equations (1) and (2):

$${a}_{x 1} = {ω}^{2} {r}_{1} , \qquad ( 1 )$$

$${a}_{x 2} = {ω}^{2} {r}_{2} , \qquad ( 2 )$$

where ω represents the angular velocity, aₓ₁ and aₓ₂ represent the radial accelerations measured by the first accelerometer (x₁) 120a and the second accelerometer (x₂) 120c, respectively, in the x-direction and r₁ and r₂ denotes radial distances of the first accelerometer 120a and the second accelerometer 120c, respectively, from a fixed reference point (such as knee joint).

Further, the angular velocity ω is calculated by subtracting the radial accelerations measured by the first accelerometer 120a and the second accelerometer 120c using equations (3) and (4).

$${a}_{x 2} - {a}_{x 1} = {ω}^{2} ( {r}_{2} - {r}_{1} ) = {ω}^{2} D , \qquad ( 3 )$$

$$ω = \sqrt{\frac{{a}_{x 2} - {a}_{x 1}}{D}} \qquad ( 4 )$$

Further, tangential accelerations may be measured in the y-direction by using equations (5) and (6):

$${a}_{y 1} = α {r}_{1} , \qquad ( 5 )$$

$${a}_{y 2} = α {r}_{2} , \qquad ( 6 )$$

where α is the angular acceleration, a_{y1} and a_{y2} represent the tangential accelerations measured by the first accelerometer 120a and the second accelerometer 120c in the y-direction.

Furthermore, the angular acceleration a is calculated by subtracting the tangential accelerations measured by the first accelerometer 120a and the second accelerometer 120c using equations (7) and (8):

$${a}_{y 2} - {a}_{y 1} = α ( {r}_{2} - {r}_{1} ) = α D , \qquad ( 7 )$$

$$α = \frac{{a}_{y 2} - {a}_{y 1}}{D} \qquad ( 8 )$$

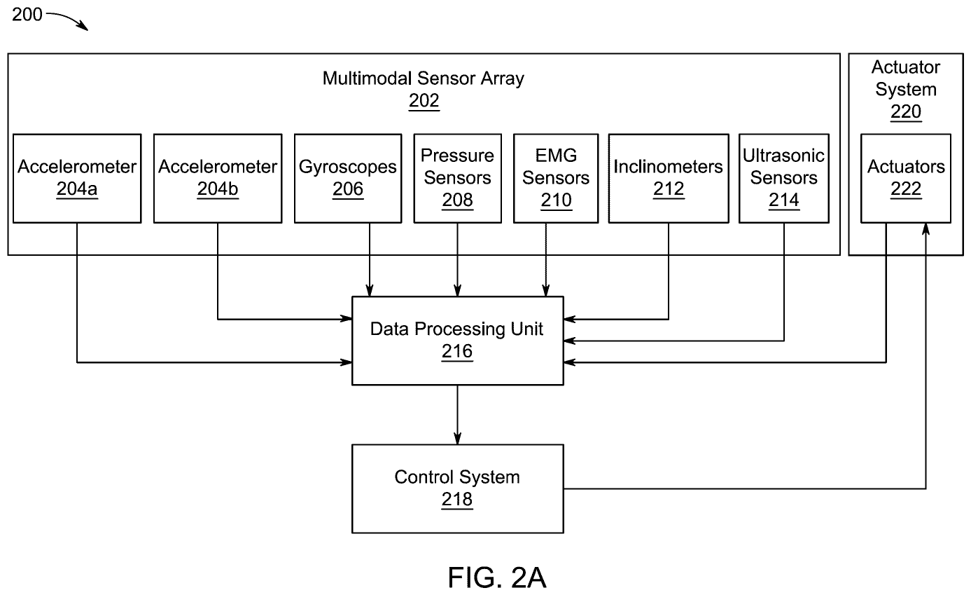

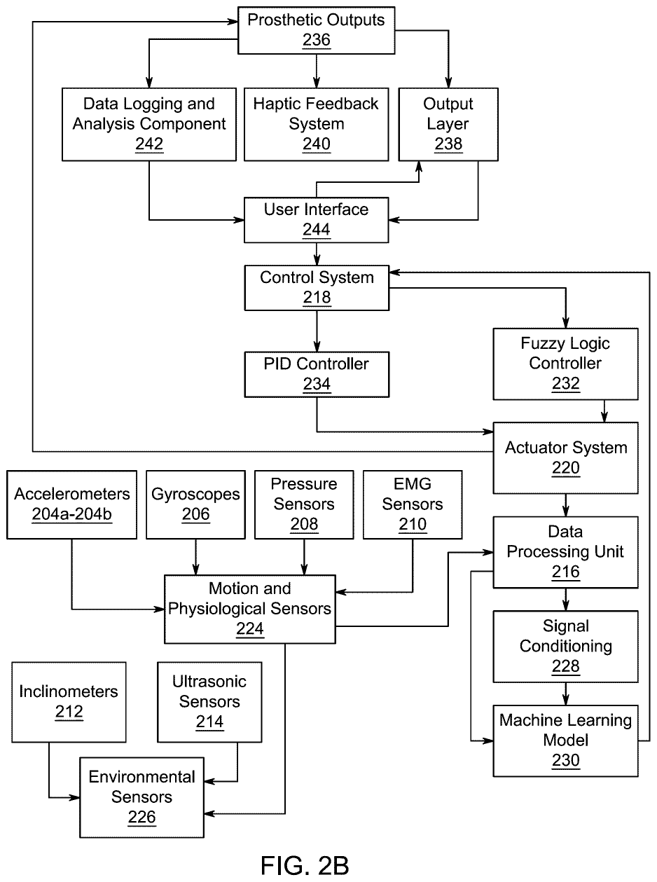

In an embodiment, an accelerometer-based method for measuring angular rotation relies on placing the two accelerometers 120a and 120c at the fixed distance (D) 128. Then, the readings of the radial accelerations and the tangential acceleration are used to compute the angular velocity and the angular acceleration. In an embodiment, accelerometer readings may enable a calculation of angular movement over a given time interval (Δt), allowing a control system 218 (as shown in FIG. 2A) of the prosthetic limb 102 to infer angle changes and motion direction from the computed angular velocity and the angular acceleration.

The accelerometer-based method may be effective for rapid rotations with significant angular accelerations, as it minimizes errors and drift while accurately determining the motion direction. However, an efficacy of the accelerometer-based method decreases when the angular accelerations are very small. In an embodiment, the microcontroller may process accelerometer data (i.e., acceleration values along the three axes) by computing difference between the accelerometer readings. For example, the microcontroller reads X-axis acceleration data from both the accelerometers 120a and 120c, one positioned at r₁ and the other at r₂. Further, the microcontroller may compute the angular velocity (@) using the difference between the readings, following equations (1) to (4). If analog accelerometers are used, the microcontroller may amplify weak signals via an operational amplifier circuit before processing.

Additionally, a Direct Current (DC)-DC converter may be used, which initially outputs 7.2V but is regulated to 5V and inverted to −7.2V using a 7805-voltage regulator and the DC-DC converter. This optimized setup may ensure high-quality accelerometer output and may eliminates a need for additional capacitors for noise filtering, resulting in a stable and noise-free signal acquisition for precise motion tracking.

FIG. 2A illustrates a block diagram of a prosthetic limb management system 200 (hereinafter referred to as the system 200), according to certain embodiments. In an embodiment, the system 200 is configured to enhance functionality and adaptability of the prosthetic limb 102 by integrating multimodal sensory data (e.g., data collected from multiple sensors) with adaptive control algorithms (e.g., algorithms that dynamically adjust the response of the prosthetic limb 102 based on the multimodal sensory data). The system 200 is also configured to autonomously adjust behavior of the prosthetic limb 102 based on real-time analysis of environmental factors, such as user input (e.g., manual adjustments, activity mode selection) and physiological signals (e.g., muscle activation or gait patterns detected through the sensors), which provides a dynamic response to mimic natural human movements accurately. The system 200 is also configured to incorporate machine learning algorithms that continuously refine the behavior of the prosthetic limb 102, adapting to user preferences and mobility requirements. Furthermore, the system 200 is configured to offer an accessible interface for customizable user settings and reduce cognitive and physical burden on the individuals.

In an embodiment, the system 200 may be embedded within the prosthetic limb 102. In another embodiment, the system 200 may be embedded in an external device (e.g., a smartphone, a tablet, a dedicated computing unit, connected through Bluetooth, Wireless Fidelity (Wi-Fi), or another communication protocol). In yet another embodiment, the system 200 may be a detachable module that may be attached or removed from the prosthetic limb 102, allowing for replacement or easy upgrades. In an embodiment, the system 200 may be embedded within the prosthetic limb 102; however, computationally intensive tasks (e.g., the adaptive control algorithms or the machine learning algorithms) may be processed in an external unit (e.g., cloud-based system).

The prosthetic limb 102 includes a multimodal sensor array 202 integrated into the prosthetic limb body 104 to obtain the sensor data. The sensor data is used to derive parameters that are indicative of a physiological state of the subject 100 and an environment in a proximity of the prosthetic limb 102. As used herein, the term “physiological state of the subject 100” refers to a current condition of the body of the subject 100, such as the muscle activity, body movements, the gait patterns, pressure and force distribution, balance and so forth. The multimodal sensor array 202 includes accelerometers 204a-204b, gyroscopes 206, pressure sensors 208, Electromyography (EMG) sensors 210, inclinometers 212 and ultrasonic sensors 214.

In an embodiment, the accelerometers 204a-204b (hereinafter referred to as the accelerometers 204) may be positioned near the knee and the ankle of the prosthetic limb 102 to measure linear acceleration and deceleration during the movement (e.g., a rapid forward movement when initiating a step and a gradual decrease in speed before foot placement). The measurement of the linear acceleration and deceleration enables real-time detection of gait phases. The gyroscopes 206 may also be positioned near the knee and the ankle of the prosthetic limb 102, to collect the angular velocity (i.e., the angular motion data), which is used to understand rotational movements. The pressure sensors 208 may be embedded in a foot sole of the prosthetic limb 102 to detect a force exerted by the subject 100, such as weight distribution while walking. The EMG sensors 210 may be mounted in thigh muscles of a residual limb of the subject 100 to collect electrical activity data, which indicates subject intent for the movement. As used herein, the term “residual limb” refers to a part of the missing limb (e.g., leg) that remains after the amputation. The inclinometers 212 may be located at the prosthetic limb knee 106 to collect inclination angle data, which is indicative of a slope (i.e., uphill or downhill), in which the subject 100 is navigating. The inclination angle data is useful for adjusting knee dynamics on varied terrains. The ultrasonic sensors 214 may be affixed on a lower part (shin) of the prosthetic limb 102 for detecting an obstacle in a path of the subject 100 to avoid collisions or guide movement.

According to an embodiment, the system 200 includes a data processing unit 216, a control system 218 and an actuator system 220. The data processing unit 216 is communicatively connected to the multimodal sensor array 202. The data processing unit 216 is configured to receive the sensor data from the multimodal sensor array 202. The data processing unit 216 is configured to process the sensor data to generate a processed sensor data. The data processing unit 216 is configured to transmit the processed sensor data to the control system 218. The data processing unit 216 includes, but not limited to, microprocessors, embedded processors, Field-Programmable Gate Arrays (FPGAs), dedicated Artificial Intelligence (AI) processors, and so forth. Embodiments of the present disclosure are intended to include or otherwise cover any type of the data processing unit 216, including known related art and/or later developed technologies. In an exemplary embodiment, the microprocessors may be capable of executing the machine learning algorithms to generate the processed sensor data.

The control system 218 is communicatively connected to the data processing unit 216 to receive the processed sensor data from the data processing unit 216. The control system 218 is configured to generate control decisions about adjustments in the movements of the prosthetic limb 102 based on the processed sensor data. In other words, the control system 218 generates a control signal for adjusting mechanical responses (i.e., movement) of the prosthetic limb 102 based on the sensor data and machine learning predictions received from the data processing unit 216. The control system 218 may include, but not limited to, a proportional myoelectric control, a microprocessor-based control, an adaptive control, a hybrid control system, and so forth. Embodiments of the present disclosure are intended to include or otherwise cover any type of the control system 218, including known related art and/or later developed technologies.

The actuator system 220 is communicatively connected to the control system 218 to receive the control decisions for executing movement adjustments in the prosthetic limb 102. The actuator system 220 is configured to convert the received control decisions into mechanical actions, ensuring precise and adaptive adjustments in the prosthetic limb 102. In an embodiment, the actuator system 220 may include embedded sensors (e.g., position sensors and force sensors) that continuously monitor movement of actuators 222 in real time. In such embodiment, the embedded sensors may provide feedback to the data processing unit 216, which analyses the feedback to detect any discrepancies between an intended movement and an actual movement. If a discrepancy is identified, the data processing unit 216 may be configured to enable the control system 218 to dynamically adjust the control decisions to optimize the movement and enhance the performance of the prosthetic limb 102.

In an embodiment, the actuator system 220 may include the actuators 222 of various types, including but not limited to, electric actuators, hydraulic actuators, pneumatic actuators, linear actuators, and rotary actuators. Embodiments of the present disclosure encompass all types of the actuators 222, including existing technologies and future advancements in actuator design and control.

FIG. 2B illustrates a schematic representation of functional components of the prosthetic limb 102, according to certain embodiments. The functional components of the prosthetic limb 102 include motion and physiological sensors 224, environmental sensors 226, the data processing unit 216, the control system 218 and the actuator system 220.

The motion and physiological sensors 224 include the accelerometers 204a-204b, the gyroscopes 206, the pressure sensors 208 and the EMG sensors 210. The accelerometers 204a-204b are configured to obtain accelerometer data. The accelerometer data is further used to derive a speed of the subject 100 and a joint angle of the prosthetic limb 102. The speed and joint angle are indicative of the gait of the subject 100. In other words, the accelerometers 204a-204b may be configured to obtain the accelerometer data by measuring the acceleration along x, y and z axis for tracking the movement of the prosthetic limb 102.

The gyroscopes 206 are configured to measure the angular motion data. The angular motion data is indicative of an angular orientation of one or more joints of the prosthetic limb 102. The angular orientation indicates a position of the prosthetic limb 102 in space and helps in stabilizing and adjusting the movements of the prosthetic limb 102. Further, the pressure sensors 208 are configured to obtain pressure data. The pressure data is indicative of the force distribution across the foot and is used to derive weight bearing and balance parameters during standing or walking of the subject 100. The EMG sensors 210 are configured to capture the electrical activity data. The electrical activity data is indicative of electrical activity produced by skeletal muscle activity, which is indicative of the subject intent for the movement. For example, if the subject 100 shifts the weight onto the prosthetic limb 102 while climbing the stairs, the pressure sensors 208 detect increased load while the gyroscopes 206 capture an angular tilt of the prosthetic limb 102.

The environmental sensors 226 include the inclinometers 212 configured for slope detection (i.e., detecting whether the subject 100 is walking uphill or downhill) and the ultrasonic sensors 214 are configured to obtain distance data, which is indicative of obstacle proximity. The inclination angle data and the distance data are then used to derive a terrain type of the environment. The motion and physiological sensors 224 and the environmental sensors 226 may be configured to transmit the sensor data to the data processing unit 216.

The data processing unit 216 is configured to collect the sensor data from the motion and physiological sensors 224 and the environmental sensors 226. The data processing unit 216 is configured to perform signal conditioning 228 on the sensor data to prepare the sensor data for analysis. The signal conditioning 228 may include filtration of the sensor data, normalization of the sensor data and feature extraction from the sensor data.

Further, the data processing unit 216 is configured to utilize a machine learning (ML) model 230 that is trained on training data to generate model predictions (i.e., processed sensor data). The training data includes sensor data, corresponding subject intent and corresponding prosthetic limb parameters. The ML model 230 outputs the processed sensor data as a predicted subject intent and predicted prosthetic limb parameters. The predicted subject intent for movement includes at least one of starting, stopping or changing direction. The prosthetic limb parameters include at least one of joint stiffness, the joint angle, damping, a motor speed, a motor torque, and so forth. In an embodiment, the data processing unit 216 is configured to determine slope data by obtaining the inclination angle data and the angular motion data for a specified period from the multimodal sensor array 202. Further, the data processing unit 216 is configured to compare the inclination angle data with specified threshold angle ranges to classify the slope of the path into one of multiple categories such as mild, moderate, and steep. The threshold angle ranges are predefined values representing different slope categories. In an exemplary embodiment, the threshold angle ranges may be determined based on biomechanical studies, safety requirements, user comfort, and so forth. For example, the mild slope ranges from 0° to 5°, the moderate slope ranges from 5° to 15° and the steep slope greater than 15°. The data processing unit 216 is further configured to determine the adjustments to the prosthetic limb parameters based on the determined category of the slope to generate adjusted prosthetic limb parameters.

Further, the data processing unit 216 is configured to determine obstacle avoidance by obtaining the distance data to potential obstacles in the path of the subject 100 from the multimodal sensor array 202. The data processing unit 216 is further configured to identify a potential obstacle based on a specified minimum safe distance. The minimum safe distance is a predefined threshold that represents a closest distance at which an obstacle is detected. In an embodiment, the data processing unit 216 may be configured to compare the distance data with the specified minimum safe distance. If the distance data is greater than the minimum safe distance, then the proximity of the obstacle is considered as far proximity, and if the distance data is less than or equal to the minimum safe distance, then the proximity of the obstacle is considered as close proximity.

The data processing unit 216 is also configured to determine an avoidance type and size of the potential obstacle. In an exemplary embodiment, the size of the potential obstacle may be determined based on a vertical height estimation (i.e., the ultrasonic sensors 214 measure the height of the obstacle by scanning different angles) and a width measurement (i.e., as the subject 100 approaches the obstacle, continuous distance measurements help calculate the width by detecting how long the object remains within sensor's detection range). The avoidance type may be “step-over” or “step-side.” The data processing unit 216 is further configured to determine the adjustments to the prosthetic limb parameters based on the avoidance type and the size of the potential obstacle to generate adjusted prosthetic limb parameters. The data processing unit 216 is configured to transmit the model predictions (i.e., processed sensor data/adjusted prosthetic limb parameters) to the control system 218.

The control system 218 is configured to generate the control signal based on the model predictions (i.e., the processed sensor data). In an embodiment, the control system 218 is configured to generate the control signal for slope navigation based on the adjusted prosthetic limb parameters. In an embodiment, the control system 218 is configured to generate the control signal for obstacle avoidance based on the adjusted prosthetic limb parameters. In at least one example embodiment, the control system 218 is configured to generate the control signal using a fuzzy logic controller 232 and a proportional-integral-derivative (PID) controller 234 of the control system 218. In an embodiment, the generated control signal has control parameters for adjusting the movement of the prosthetic limb 102. The control parameters include values of the prosthetic limb parameters to be adjusted.

In an embodiment, the control signal is a combination of a first control signal generated by the fuzzy logic controller 232 and a second control signal generated by the PID controller 234. The fuzzy logic controller 232 is configured to generate the first control signal for adjusting the prosthetic limb parameters based on the current position or movement of the prosthetic limb 102, which may be detected using the accelerometers 204 and the gyroscopes 206.

In an exemplary embodiment, the fuzzy logic controller 232 may generate the first control signal by analyzing the current position and movement of the prosthetic limb 102 using one or more predefined fuzzy rules. The fuzzy logic controller 232 receives data associated with the angular movement from the accelerometers 120a-120d mounted on the unaffected leg 110 of the subject 100 and maps the data to the gait cycle phases. The fuzzy logic controller 232 further maps the gait cycle phases to the corresponding prosthetic limb parameters, ensuring synchronized movement. The fuzzy logic controller 232 further performs defuzzification to convert fuzzy outputs (i.e., different adjustments of the prosthetic limb parameters based on the gait cycle phases) into the control signal that is encoded to adjust the prosthetic limb parameters accordingly.

The PID controller 234 is configured to generate the second control signal to reduce any error between an actual position and a desired position of the prosthetic limb 102. In an embodiment, the desired position of the prosthetic limb 102 is set based on predefined gait parameters (i.e., step length, step time, joint stiffness, and so forth) or the subject intent and the actual position is measured using the embedded sensors. The PID controller 234 fine-tunes the position and the speed of the prosthetic limb 102 for a smooth operation. In an exemplary embodiment, the PID controller 234 continuously monitors a position error that is calculated as a difference between the desired position and the actual position of the prosthetic limb 102. The second control signal may be computed using three components such as a Proportional (P) component, which is directly proportional to the error and provides immediate correction, an Integral (I) component which accumulates past error over time to eliminate steady-state errors, and a Derivative (D) component which predicts future errors by considering a rate of change of error, thus improving stability and responsiveness. By dynamically adjusting the components, the PID controller 234 fine-tunes the position and the speed of the prosthetic limb 102, ensuring smooth operation of the prosthetic limb 102.

In an embodiment, combined output from the components may generate the second control signal that dynamically adjusts the prosthetic limb parameters. The PID controller 234 continuously updates the control signal in real-time to adapt to changing conditions, optimizing stability and responsiveness during the movement. The control system 218 is configured to transmit the generated control signal to the actuator system 220.

The actuator system 220 is configured to enable the actuators 222 to physically adjust the prosthetic limb 102 by adjusting the prosthetic limb parameters based on the control parameters received in the control signal. In other words, the actuator system 220 receives the control signal from the control system 218 and transmits the control signal to the actuators 222 (such as motors, servos or hydraulic systems). The control signal encodes how much movement is needed at specific joints (e.g., knee flexion or ankle rotation). The actuators 222 may be mechanical force generators configured to physically adjust the prosthetic limb 102, such as bending the knee, lifting the foot or adjusting the orientation of the prosthetic limb 102. The actuator system 220 ensures that the movements of the prosthetic limb 102 are precise, smooth and align with real-time requirements of the user's movements and the environment.

To maintain movement accuracy and responsiveness, the actuator system 220 is also configured to transmit the feedback to the data processing unit 216 for real-time monitoring and adjustments. In an embodiment, the feedback may include the sensor data from the embedded sensors, force sensors, and encoders that track the actual movement, force exerted, and the joint angles of the prosthetic limb 102. The data processing unit 216 is configured to analyze the sensor data to identify any discrepancies between the intended movement (as per the control signal) and the actual movement executed by the actuators 222. If the data processing unit 216 is configured to detect an error, such as an insufficient knee bend, excessive torque, or misalignment, then the data processing unit 216 is configured to enable the control system 218 to adjust the control parameters in subsequent control signals. Additionally, a feedback loop allows the prosthetic limb 102 to adapt to changing terrain, user walking patterns, and dynamic environmental factors, ensuring a smooth and natural gait cycle.

In an embodiment, the actuator system 220 may be configured to generate prosthetic outputs 236 that include real-time adjustments in the prosthetic limb parameters. In an exemplary embodiment, the prosthetic outputs 236 may be generated by the actuators 222 based on the control signal. Once the prosthetic outputs 236 are generated by the actuators 222, the prosthetic outputs 236 may be transmitted to an output layer 238, a haptic feedback system 240 and a data logging and analysis component 242.

The output layer 238 may be configured to display the real-time status of the prosthetic limb 102, including movements, adjustments, alerts, issues or errors, on the user interface 244. For example, if the knee is not responding correctly or maintenance is needed, the feedback may be displayed on the user interface 244.

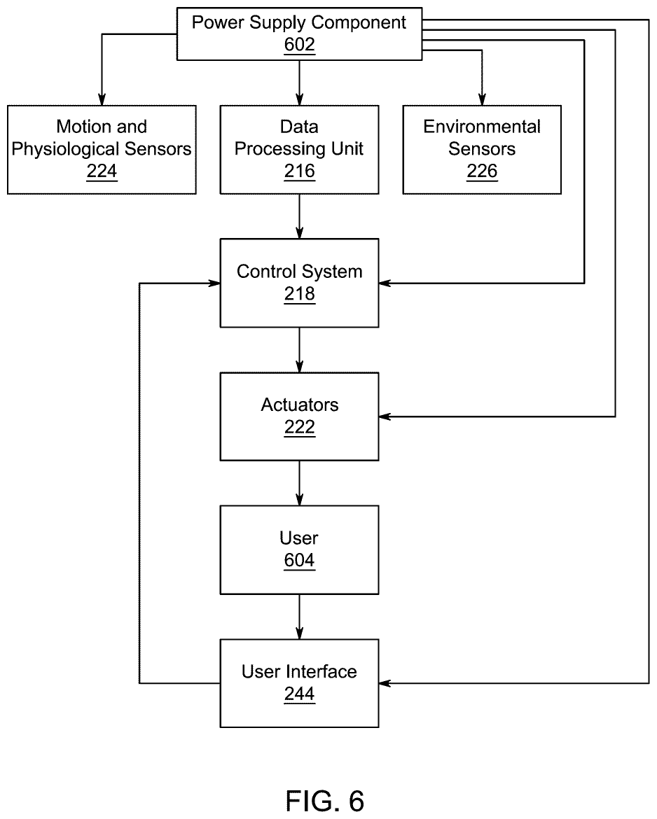

The haptic feedback system 240 may be configured to extract data regarding the position and the movement of the prosthetic limb 102 from the prosthetic outputs 236. The haptic feedback system 240 is configured to provide haptic feedback regarding positioning and the movement of the prosthetic limb 102 to a user 604 (as shown in FIG. 6). In an exemplary embodiment, the haptic feedback system 240 is configured to provide the user 604 with tactile sensations that reflect a current state of the prosthetic limb 102. For instance, when the knee bends, the user 604 may feel vibrations or resistance changes that inform the user 604 about the status of the prosthetic limb 102.

In an embodiment, the data logging and analysis component 242 may be configured to continuously log the prosthetic outputs 236, including the movement patterns, the sensor data, and actuator adjustments. The logged prosthetic outputs 236 may be stored in an embedded memory within the prosthetic limb 102 for further analysis and diagnostic purposes. In an embodiment, the data logging and analysis component 242 may also be equipped with analytical tools to analyse the performance of the prosthetic limb 102, detect anomalies, and predict maintenance needs, thereby enhancing the longevity and reliability of the prosthetic limb 102. The data logging and analysis component 242 may be configured to transmit the analysed data to the user interface 244, allowing the user 604 to monitor real-time prosthetic performance and review diagnostics of the prosthetic limb 102.

The user interface 244 may provide options for customizing prosthetic settings based on activity level, terrain conditions, or comfort preferences. In an embodiment, the user interface 244 may be accessed via a mobile application or a desktop interface, providing the user 604 with an intuitive way to customize the behavior of the prosthetic limb 102.

In an embodiment, the prosthetic settings may be transmitted from the user interface 244 to the control system 218, which then adjusts the control parameters for the actuator system 220 to align the prosthetic outputs 236 with the user's requirements. For instance, if the user 604 specifies that the knee should flex more easily or apply more resistance during walking, the control system 218 fine-tunes the fuzzy logic controller 232 and the PID controller 234 to match the user's requirements. Further, the control system 218 may be configured to transmit updated control signals to the actuator system 220, making the necessary adjustments to the performance of the prosthetic limb 102.

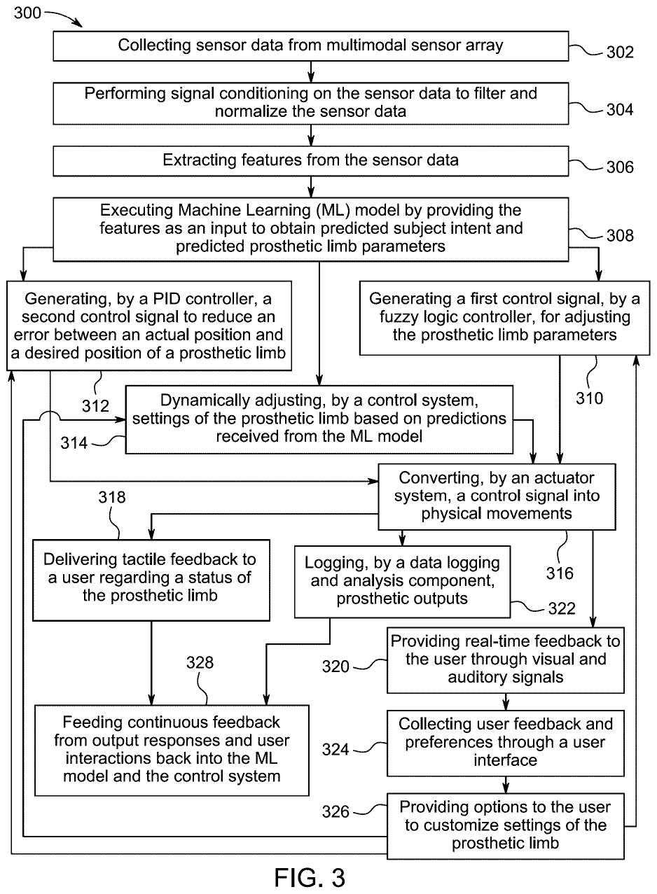

FIG. 3 illustrates a flowchart of a process 300 representing a detailed visualization for controlling the prosthetic limb 102, according to certain embodiments.

At step 302, the process 300 includes collecting the sensor data from the multimodal sensor array 202, which includes the multiple sensors (i.e., the motion and physiological sensors 224 and the environmental sensors 226) capturing different physiological and environmental parameters. This step includes activating all the sensors to measure parameters, such as the orientation of the prosthetic limb 102, pressure distribution, muscle activity, joint angles, and terrain characteristics. This step also includes synchronizing all the sensors to ensure accurate time-aligned sensor readings. In an exemplary embodiment, the sensors in the multimodal sensor array 202 may be connected to a power supply component 602 (as shown in FIG. 6) that supplies power to the sensors, which in turn activates the sensors, ensuring that the sensors are powered and operational for data collection.

At step 304, the process 300 includes performing the signal conditioning 228, by the data processing unit 216, on the sensor data to prepare the sensor data for analysis. This step includes filtering the sensor data to remove noise and distortion to generate filtered sensor data. This step further includes normalizing the filtered sensor data to generate normalized sensor data having a consistent range or format across different sensors of the multimodal sensor array 202. In an exemplary embodiment, the filtering of the sensor data includes applying signal processing techniques such as, but not limited to, low-pass filters, high-pass filters, band-pass filters, and so forth to remove the noise, the distortion, and unwanted frequency components from the sensor data. After filtering, the normalization may be performed to standardize the filtered sensor data. The normalization includes scaling values of the sensor data to a fixed range (e.g., min-max normalization) or adjusting the values of the sensor data based on statistical properties like mean and standard deviation (z-score normalization). The signal conditioning 228 of the sensor data ensures consistency in the sensor readings, enabling accurate comparisons and integration of the multimodal sensor data.

At step 306, the process 300 includes extracting features, by the data processing unit 216, from the sensor data. The features are indicative of subject movement patterns and environmental conditions. The subject movement patterns are derived from the sensor data and are indicative of aspects such as the walking speed of the subject 100, which may be determined by analyzing the gait dynamics. Additionally, the features related to the environmental conditions are extracted, including the terrain type, which may be inferred from the sensor data like the pressure distribution or inclination angles, and obstacle proximity. The features provide a comprehensive view of the subject's movement behavior and the surrounding environment, enabling the prosthetic limb 102 to adapt and optimize control strategies based on real-time movement and terrain conditions. In an exemplary embodiment, various signal processing techniques, such as, but not limited to, Fourier transforms, wavelet analysis, and the machine learning algorithms, may be used to detect the movement patterns, classify the terrain types, and predict changes in movement dynamics. The extracted features provide insights for optimizing control of the prosthetic limb 102 (i.e., how the prosthetic limb 102 moves and responds to user's actions, ensuring smooth, natural, and efficient movement) and adaptive response mechanisms (i.e., prosthetic limb's ability to automatically adjust its behavior based on changing conditions, such as variations in the walking speed, the terrain type, or obstacle presence).

At step 308, the process 300 includes executing, by the data processing unit 216, the ML model 230 by providing the features as an input to obtain the predicted subject intent and the predicted prosthetic limb parameters. In an exemplary embodiment, the ML model 230 predicts the subject intent, such as whether the subject 100 intends to walk, stop, adjust the gait, or navigate the obstacles, and the prosthetic limb parameters by analyzing the extracted features from the sensor data. In an embodiment, the features, which represent the subject movement patterns and the environmental conditions, may be fed into the trained ML model 230. The trained ML model 230 utilizes historical data and learned patterns to classify the subject intent, such as standing, walking, or transitioning between the different gait phases. Simultaneously, the trained ML model 230 predicts the optimal prosthetic limb parameters to ensure smooth and adaptive movement. By continuously refining predictions based on real-time sensor data, the ML model 230 enhances the responsiveness and stability of the prosthetic limb 102.

At step 310, the process 300 includes generating the first control signal, by the fuzzy logic controller 232, for adjusting the prosthetic limb parameters based on the current position or the movement of the prosthetic limb 102.

At step 312, the process 300 includes generating, by the PID controller 234, the second control signal to reduce the error between the actual and desired position of the prosthetic limb 102. This step includes compensating for ground reaction forces during the gait cycle phase by providing additional torque adjustments to the prosthetic limb 102. For example, when the prosthetic limb 102 makes contact with the ground during the stance phase 1002, the ground reaction forces act against the prosthetic limb 102, potentially causing instability or deviations from the expected movement. To counteract this, the PID controller 234 processes the pressure data and dynamically modifies knee torque output to maintain balance and prevent excessive flexion or collapse.

At step 314, the process 300 includes dynamically adjusting, by the control system 218 (i.e., an adaptive control system), settings of the prosthetic limb 102 based on the predictions received from the machine learning model 230. In an embodiment, the control system 218 dynamically adjusts the control strategies of the prosthetic limb 102 by continuously monitoring the sensor data, analyzing the movement patterns, and predicting the necessary adjustments in real time. In an exemplary embodiment, the control system 218 processes the input from the multimodal sensor array 202 to assess the gait dynamics, terrain changes, and the subject intent. Using the machine learning model 230, the control system 218 anticipates upcoming movements and proactively adjusts the prosthetic limb parameters to ensure a smooth and natural gait. When the movement patterns deviate from expected movement patterns, the control system 218 fine-tunes the control parameters using a reinforcement learning or adaptive feedback loops, ensuring seamless transitions between walking states such as level ground, inclines, stairs, and sudden stops. Through continuous learning and self-optimization, the prosthetic limb 102 adapts dynamically to the changing conditions, enhancing the mobility and responsiveness over time.

At step 316, the process 300 includes converting the control signal into physical movements by adjusting the mechanical components like the motors and the joints of the prosthetic limb 102. In an exemplary embodiment, once the machine learning model 230 predicts the necessary adjustments of the prosthetic limb parameters based on the sensor data, the control system 218 generates the control signal corresponding to the adjustments of the prosthetic limb parameters. The control signal may be transmitted to the actuator system 220, which regulates the operation of the actuator 222 inside the prosthetic limb 102. The actuators 222 may respond by modulating the resistance, the torque, and the angular movement, allowing the prosthetic limb 102 to adapt dynamically to terrain variations, the walking speed, and the subject intent.

At step 318, the process 300 includes delivering tactile feedback to the user 604 regarding the status of the prosthetic limb 102, enhancing user's perception and control over the prosthetic limb 102. This step is achieved through haptic actuators, such as, but not limited to, vibration motors, pressure pads, or electrical stimulation units embedded within a prosthetic socket or worn on the residual limb.

At step 320, the process 300 includes providing real-time feedback to the user 604 through visual and auditory signals through the user interface 244 such as, but not limited to, the mobile application, a smartwatch display, or onboard LED indicators. As the prosthetic limb parameters are continuously adjusted, the control system 218 transmits status updates and alerts to the user. For instance, a color-coded LED system may indicate different states, such as green for normal operation, yellow for minor adjustments, and red for critical alerts. Similarly, a companion mobile application displays detailed movement analytics, battery levels, terrain adaptation status, and gait performance insights. In an embodiment, the auditory signals, such as beeps or voice prompts, may notify the user 604 about system changes, warnings, or terrain adaptations.

At step 322, the process 300 includes logging, by the data logging and analysis component 242, the prosthetic outputs 236, including the movement patterns, the sensor readings, and the actuator adjustments.

At step 324, the process 300 includes collecting user feedback and preferences through the user interface 244, which refines the performance of the prosthetic limb 102 and user experience.

At step 326, the process 300 includes enabling the user 604 to interact directly with the prosthetic limb 102 through the user interface 244, which provides options to customize the settings and optimize the performance. In an embodiment, the user 604 may provide a manual input through the mobile application to adjust the settings like knee stiffness, damping sensitivity, and walking modes.

At step 328, the process 300 includes feeding continuous feedback from output responses and user interactions back into the machine learning model 230 and the control system 218. In an embodiment, a loop of the continuous feedback enables ongoing learning and adaptation, improving the accuracy of the prosthetic limb 102 and the responsiveness over time.

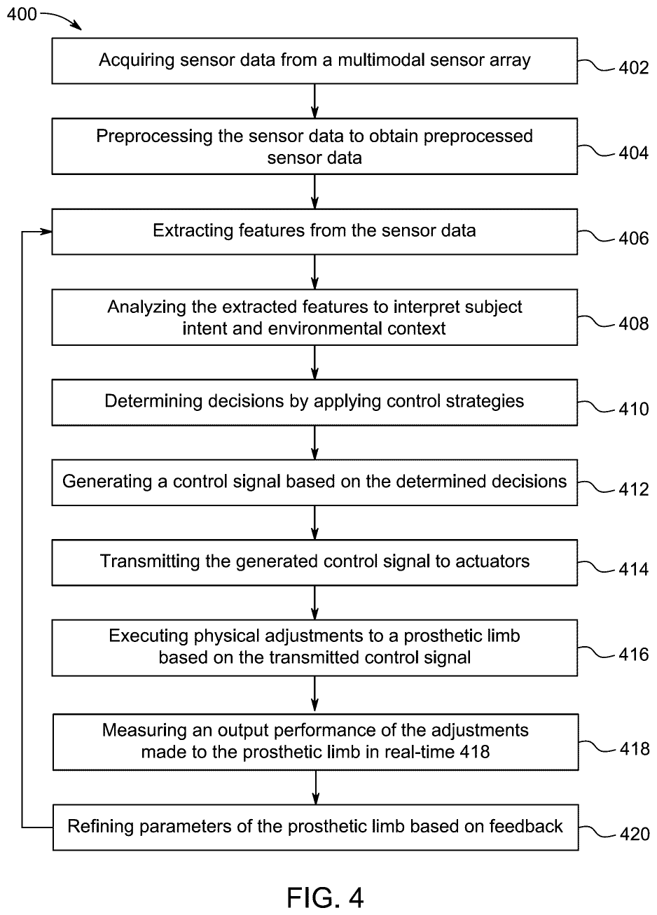

FIG. 4 illustrates a flowchart of a method 400 for adaptive control of the prosthetic limb 102 using multi-sensor integration, according to certain embodiments. The method 400 provides a comprehensive approach to controlling the prosthetic limb 102, by using real-time sensor data, the adaptive algorithms and the feedback loops to ensure smooth and efficient movement.

At step 402, the method 400 includes acquiring the sensor data from the multimodal sensor array 202 integrated into the prosthetic limb 102. The sensor data may be the accelerometer data (i.e., angular velocity), the gyroscope data (i.e., angular orientation), pressure sensor data (i.e., force distribution), EMG sensor data (i.e., muscle activity) and environmental sensor data (i.e., terrain and obstacle data).

At step 404, the method 400 includes preprocessing (i.e., filtering and normalizing), by the data processing unit 216, the sensor data to obtain the preprocessed sensor data.

At step 406, the method 400 includes extracting, by the data processing unit 216, the features from the sensor data. This step includes identifying the gait patterns, detecting any irregularities that may indicate a need for adjustment, and measuring kinematic parameters relevant to movement analysis. In an exemplary embodiment, a way the user 604 moves, including factors such as a stride length (i.e., a distance covered by a same foot in one complete gait cycle), the walking speed, and cadence (i.e., number of steps taken per minute), may be used to detect the gait patterns. By analyzing the gait patterns, the data processing unit 216 may identify any anomalies (e.g., limping or inconsistent steps) or irregularities, such as limping or inconsistent steps (e.g., uneven gait due to fatigue or discomfort). Further, the terrain type, such as a flat ground, a gravel, or the stairs, affects how the user 604 adjusts their gait, which may lead to changes in the kinematic parameters like knee flexion or a step length. The obstacle proximity also influences how the user 604 adapts their movement to avoid or navigate around the obstacles, potentially altering a step frequency or requiring adjustments in the knee stiffness.

At step 408, the method 400 includes analyzing, by the data processing unit 216, the extracted features to interpret the subject intent and the environmental context. The subject intent refers to the actions or movements the subject 100 intends to make, such as walking, stopping, adjusting speed, or navigating the obstacles. The environmental context refers to factors like the terrain type (e.g., flat ground, incline, rough terrain) or the proximity of obstacles that influence the subject's movements. By analyzing the sensor data related to the terrain (such as pressure patterns or inclination), the prosthetic limb 102 understands the current environment and may adjust the prosthetic limb's behavior accordingly.

At step 410, the method 400 includes determining, by the data processing unit 216, decisions by applying the control strategies. The control strategies are dynamically adjusted based on analyses and predictions, considering real-time factors such as the subject intent, environmental changes, and terrain variations. This allows the prosthetic limb 102 to adapt to changing conditions, enhancing its accuracy, responsiveness, and overall performance during various activities and environments.

At step 412, the method 400 includes generating, by the control system 218, the control signal based on the determined decisions.

At step 414, the method 400 includes transmitting, by the control system 218, the generated control signal to the actuators 222. The control signal commands the actuators 222 to execute motor actions that adjust the mechanical components of the prosthetic limb 102 accordingly.

At step 416, the method 400 includes executing, by the actuators 222, the physical adjustments to the prosthetic limb 102 based on the transmitted control signal. The actuators 222 physically adjust the prosthetic limb 102 to match computed requirements based on the user's movement and the environmental conditions. For instance, if the prosthetic limb 102 detects a change in the walking speed or encounters uneven terrain, the joint stiffness and the damping may be adjusted to maintain balance and support. These adjustments are continuously refined in real-time to match the ground reaction forces. The ground reaction forces refer to the forces the prosthetic limb 102 encounters when making contact with the ground and to align with the user's gait pattern, ensuring smooth, stable movement.

At step 418, the method 400 includes measuring an output performance of the adjustments made to the prosthetic limb 102 in real-time. This step includes continuously tracking key performance metrics, such as the joint angles, gait symmetry, the movement speed, and the stability of the prosthetic limb 102. This step further includes comparing the key performance metrics with expected outcomes, which may be derived from pre-established models or historical data about the user's movement patterns and desired walking behavior. In an embodiment, a comparison result may identify the discrepancies, such as a misalignment in the position of the prosthetic limb 102, inadequate knee stiffness, or imperfect damping adjustments. The discrepancies indicate whether the adjustments are effective or need further refinement. In addition to the comparison, the method 400 includes gathering the user feedback through various means, such as the manual input through the user interface 244, real-time satisfaction ratings, and so forth. The user feedback provides valuable insights into the comfort, natural feel, and overall effectiveness of the prosthetic adjustments. By analyzing both objective performance data and subjective user feedback, the adjustments of the prosthetic limb 102 may be fine-tuned continuously, ensuring that the adjustments are optimized to meet both user's functional needs and personal preferences.

At step 420, the method 400 includes refining the parameters of the prosthetic limb 102 based on the feedback gathered from the step 418. This step involves updating the control algorithms (such as, the fuzzy logic controller 232 and the PID controller 234) to align with the user's requirements, enhancing learning models (i.e., the machine learning model 230) to improve the predictions and the prosthetic outputs 236, and adjusting sensory thresholds to detect and react to the subject's movement and the environmental conditions.

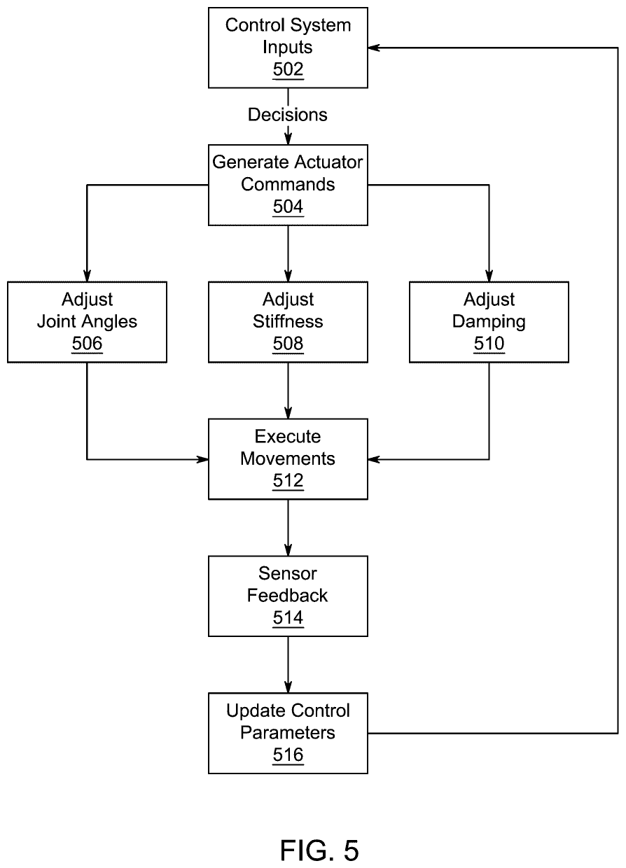

FIG. 5 illustrates an exemplary flowchart of a method 500 representing output adjustment mechanisms in the prosthetic limb 102, according to certain embodiments.

At step 502, the method 500 includes processing the sensor data by the control system 218. This step further includes using the processed sensor data, by the control system 218, to generate the control signal.

At step 504, the method 500 includes generating, by the control system 218, actuator commands based on the control signal. The actuator commands are specific instructions to the actuators 222 in the prosthetic limb 102 to adjust the prosthetic limb parameters in real-time.

At step 506, the method 500 includes generating the actuator command to adjust the joint angles to better align with the natural gait cycle of the user 604 or to adapt to the walking surface. For example, in a stance phase 1002, the prosthetic limb knee 106 may be in an extended position to support the user weight and provide stability. During the swing phase 1004, the knee joint angle may be changed to allow for smoother leg motion and to clear the ground during walking. In an exemplary embodiment, the actuators 222 may apply force to adjust the knee angle based on the actuator commands, which ensures that the knee's movement mimics a natural rhythm and motion of walking or other activities.

At step 508, the method 500 includes generating the actuator command to adjust the stiffness of the knee joint to accommodate different types of movement, such as walking, running, or climbing stairs. For instance, high stiffness is required during the stance phase 1002 to provide the support and stability when the user 604 is bearing weight. Lower stiffness is applied during the swing phase 1004 or in activities like running, where greater flexibility and smoothness of movement are required.

At step 510, method 500 includes generating the actuator command to adjust the damping setting that controls the ability of the knee to absorb shocks and control a rate of movement during transitions between the stance phase 1002 and the swing phase 1004. By adjusting the damping setting, the prosthetic limb knee 106 improves the comfort and stability when walking on varied surfaces like ramps and stairs. For example, damping is increased to control the rate of movement during weight-bearing phases to prevent jerky movements and reduce shock at heel strike or toe-off.

At step 512, the method 500 includes executing, by the actuators 222, the movement adjustments based on the actuator commands generated by the control system 218. This step includes sending, by the control system 218, the control signal to the actuators 222, which directs the actuators 222 to adjust the mechanical components of the prosthetic limb 102, such as the joint stiffness, the damping, and the position of the prosthetic limb 102.

At step 514, the method 500 includes providing, by the embedded sensors, real-time feedback about the outcomes of the adjustments. The feedback may include a joint position (e.g., did the knee reach the desired angle), the pressure distribution (e.g., was the ground contact pressure evenly distributed), and so forth.

At step 516, the method 500 includes updating the parameters of the control system 218 and decision-making processes using the real-time feedback. The parameters may include joint angle targets, stiffness settings, damping settings, and so forth. By refining the parameters in response to real-time feedback, the control system 218 enhances the adaptability and the responsiveness. This continuous loop of adjustment and feedback ensures that the prosthetic limb 102 remains highly responsive to the user's activities, providing improved comfort, efficiency and stability.

FIG. 6 illustrates a component interaction diagram 600 for the prosthetic limb 102, according to certain embodiments. The prosthetic limb 102 includes a power supply component 602 configured to supply power to electronic and mechanical components such as the sensors of the multimodal sensor array 202, the data processing unit 216, the control system 218, the actuators 222, and the user interface 244. In an embodiment, the power supply component 602 may be housed within the prosthetic body 104 (i.e., knee, ankle, calf), depending on a design of the prosthetic limb 102. In an exemplary embodiment, the power supply component 602 may be placed within a socket or a lower limb portion of the prosthetic limb 102, where the electronic components are housed. In another embodiment, the power supply component 602 may be located near the actuators 222 as the actuators 222 require a substantial amount of power. Therefore, the power supply component 602 needs to be positioned near the actuators 222 to deliver the power effectively. In yet another embodiment, the power supply component 602 may be located towards the back of the calf or below the knee, where the power supply component 602 is easily accessed for maintenance or charging. In another embodiment, the power supply component 602 may be integrated into the prosthetic limb ankle 108.

In an embodiment, the power supply component 602 may be a battery that provides the power to the components of the prosthetic limb 102. The battery may be, but not limited to, a dry battery, a rechargeable battery, and so forth. Embodiments of the present disclosure are intended to include or otherwise cover any type of the battery, including known, related art, and/or later developed technologies.

In another embodiment, the power supply component 602 may be an external power supply unit that may be, but not limited to, an Alternating Current (AC) power supply unit, a Direct Current (DC) power supply unit, and so forth. In such an embodiment, the prosthetic limb 102 may be provided with a power cord that may be used to supply the power to the components of the prosthetic limb 102. The power cord may have a plug at a first end and a connector at a second end. The plug may be inserted into a wall socket to receive the power, and the connector may be inserted into a charging port of the prosthetic limb 102 to supply the power to the components of the prosthetic limb 102. The plug of the power cord may be of any type, such as, but not limited to, a type A, a type B, a type C, and so forth. Embodiments of the present disclosure are intended to include or otherwise cover any type of the plug, including known, related art, and/or later developed technologies.

In an embodiment, the power supply component 602 may be integrated with energy management strategies to optimize battery life. For example, power consumption may be dynamically adjusted based on real-time activity, such as reducing power usage during periods of inactivity or low-motion states. Additionally, continuous battery status monitoring may enable adaptive power distribution to prevent excessive battery drain and extend operational duration. Other energy management strategies, such as regenerative braking to harvest energy during deceleration phases or low-power sleep modes for non-essential components, may further enhance battery longevity.

In an embodiment, the prosthetic body 104 may include a switch (not shown) to be operated for activating or deactivating the components of the prosthetic limb 102. In other words, the switch may be activated by the user 604 to enable the power supply component 602 to supply the power to the components of the prosthetic limb 102. In another embodiment, the switch may be deactivated by the user 604 for disabling the power supply component 602 to stop supplying the power to the components of the prosthetic limb 102. The switch may be of any type, such as, but not limited to, a toggle switch, a touch switch, and the like. Embodiments of the present disclosure are intended to include or otherwise cover any type of the switch, including known, related art, and/or later developed technologies. In an exemplary embodiment, the power may start flowing through dedicated wires to supply the power to the components of the prosthetic limb 102 when the switch is turned on by the user.

In an exemplary embodiment, the power supply component 602 provides the power to the components of the prosthetic limb 102 through a power distribution network. The power distribution network includes a voltage regulator, power management circuits and the dedicated wiring that routes the power to the components of the prosthetic limb 102. In an embodiment, the voltage regulator may be used to convert a power voltage into a level required by the different sensors. For example, some sensors may operate at 3.3V, while other sensors may operate at 5V. Further, the power distribution network routes the regulated power to the sensors through the dedicated wires. In an embodiment, the power distribution network may be integrated within the control system 218. In another embodiment, the power distribution network may be housed within the prosthetic limb knee 106, where batteries and control electronics are located.

Upon receiving the power, the components of the prosthetic limb 102 may be activated to perform corresponding functions. For example, the motion and physiological sensors 224 and the environmental sensors 226 may be activated to obtain the sensor data. The motion and physiological sensors 224 and the environmental sensors 226 may further transmit the sensor data to the data processing unit 216. The data processing unit 216 may be activated to receive the sensor data from the motion and physiological sensors 224 and the environmental sensors 226 and process the sensor data to generate the processed sensor data.

The data processing unit 216 transmits the processed sensor data to the control system 218 that is activated to generate the control signal based on the processed sensor data. The control system 218 transmits the generated control signal to the actuators 222. The actuators 222 may be activated to physically adjust the prosthetic limb 102 by adjusting the prosthetic limb parameters based on the control signal. In an embodiment, the user interface 244 may also be activated to enable the user 604 to provide the feedback on the prosthetic limb 102.

### Test Analysis

To evaluate the performance of the prosthetic limb 102, a simulated testing environment may be established that replicates real-world conditions to assess the adaptability of the prosthetic limb 102. The simulated testing environment includes a variable terrain platform, a treadmill with adjustable speed and incline and an instrumented gait analysis system. The variable terrain platform may be a mechanically controlled platform that simulates various terrains such as flat surfaces, slopes, and irregular, obstacle-laden paths. For example, the variable terrain platform mimics how the prosthetic limb 102 should respond on a gravel path, where stability and traction are essential. Similarly, the variable terrain platform mimics walking on rough, uneven surfaces, such as dirt or grass, which require the prosthetic limb 102 to adapt its flexibility and response time.

The treadmill allows for assessing the prosthetic outputs 236 for changes in the walking speed and incline. For instance, the treadmill helps in testing the prosthetic's ability to adapt when the user 604 walks faster or changes an incline angle, simulating activities like ascending stairs or walking uphill. The instrumented gait analysis system captures detailed biomechanical data, such as the joint angles, forces exerted, and the acceleration during movement. For example, the instrumented gait analysis system measures the torque and angular displacement of the knee, ensuring that the prosthetic limb 102 mimics the natural movement during various activities.

Test Scenarios: Multiple test scenarios may be designed to assess the performance of the prosthetic limb 102 under various conditions. For example, in a flat terrain walking scenario, the subject 100 may walk on a flat surface at different speeds. The goal is to evaluate how well the prosthetic limb 102 maintains the stability and the comfort at varying walking speeds, ensuring smooth transitions between strides and effective damping adjustments for a comfortable walking experience. In a slope navigation scenario, the subject 100 may navigate upward and downward slopes. This scenario evaluates the ability of the prosthetic limb 102 to adjust the joint stiffness and the damping to maintain the balance and energy efficiency. For example, when walking uphill, the prosthetic limb 102 may increase a torque output to provide more support, while walking downhill requires the adjustments to prevent excessive forward bending. In an obstacle avoidance scenario, the response of the prosthetic limb 102 to sudden obstacles is tested. The subject 100 may encounter the obstacles like a curb or small objects, and the ultrasonic sensors 214 detect the obstacles. The control system 218 then adjusts the prosthetic limb 102 to either step over or navigate around the obstacle smoothly, ensuring the user's motion remains uninterrupted.

Test Results: The performance of the prosthetic limb 102 was evaluated based on the test scenarios, and test results demonstrated its ability to adapt dynamically to different walking conditions.

In the flat terrain walking scenario, the prosthetic limb 102 demonstrated excellent stability and comfort, with its damping characteristics dynamically adjusting to match a walking pace, leading to smooth gait transitions. The prosthetic limb 102 seamlessly adapts to changes in the walking speed, allowing for an efficient walking experience on the flat surfaces. During the slope navigation scenario, the prosthetic limb 102 adjusts the torque output and the angular position as the incline changes, helping the subject 100 ascend and descend the slopes without strain. In the obstacle avoidance scenario, the ultrasonic sensors 214 successfully detect the obstacles, and the control system 218 quickly computes a strategy to navigate around them, maintaining safety and ensuring continuous motion without interruption. These test results demonstrate the prosthetic's capability to adapt dynamically to different walking conditions which further enhance the user's mobility.

In an embodiment, performance metrics from testing in both simulated and real-world environments further demonstrate the effectiveness of the prosthetic limb 102. For example, stability and comfort metrics indicate a 40% improvement in the stability and user-reported comfort, particularly during the slope navigation and the obstacle avoidance. Energy efficiency tests show a 30% reduction in energy consumption compared to traditional active prosthetics, mainly due to optimized motor control and adaptive energy management system. Additionally, user satisfaction survey reports an 85% satisfaction rate, with users highlighting the enhanced mobility and intuitive control features of the prosthetic limb 102.

The tests conducted in the simulated environment validate the advanced capabilities of the prosthetic limb 102, proving the potential of the prosthetic limb 102 to greatly improve mobility for lower-limb amputees. By integrating sensing technologies, adaptive control algorithms, and a user-centered design, the prosthetic limb 102 surpasses current standards in prosthetic technology. The prosthetic limb 102 offers unprecedented functionality, adaptability, and autonomy, providing the users with an enhanced and more natural experience while walking or performing various tasks.

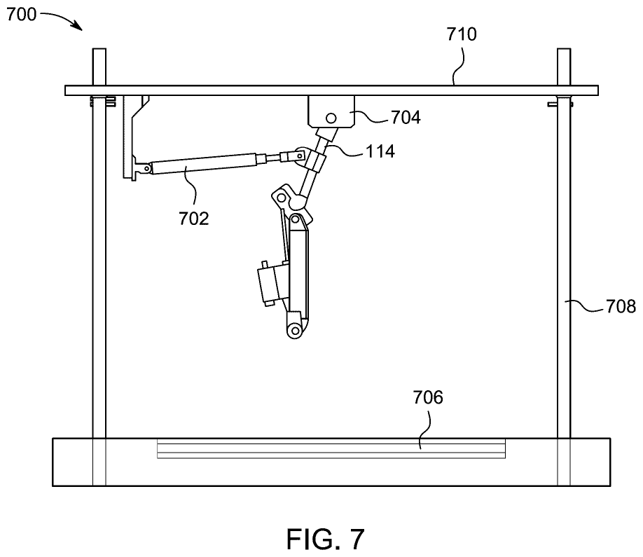

FIG. 7 illustrates a treadmill-based prosthetic limb testing system 700, according to certain embodiments. The testing system 700 simulates real-world walking conditions. The testing system 700 includes a pneumatic piston 702 that simulates a hip motion, providing a controlled actuation to mimic natural leg movement. The pneumatic piston 702 may be operated using compressed air to extract and retract, thereby moving the prosthetic limb 102 through a controlled range of motion. The pneumatic piston 702 may adjust the force and the speed of movement, allowing the testing system 700 to test how the prosthetic limb 102 adapts to various walking speeds and the ground reaction forces.

The testing system 700 includes a hip joint 704 that serves as an attachment point for the prosthetic limb 102, enabling the rotational movement similar to a biological hip. The hip joint 704 connects the pneumatic piston 702 to the femur 114, allowing force transmission. In an embodiment, the hip joint 704 may include motion sensors to track a hip angle, the speed and the acceleration during the movement.

The femur 114 represents an upper leg segment of the prosthetic limb 102. The femur 114 serves as a structural link between the hip joint 704 and the prosthetic limb knee 106. The femur 114 is designed to support weight distribution and force transmission to the knee and lower limb components.

The testing system 700 includes a treadmill 706, which provides a moving surface, allowing the prosthetic limb 102 to experience different walking conditions. For example, the treadmill 706 may be programmed to simulate different walking speeds, inclines and terrain types. In an embodiment, the force sensors may be embedded in the treadmill 706 to measure the ground reaction forces and the gait patterns.

The testing system 700 also includes a Controlled Rotational Shaft (CRS) 708, which may be a structural component supporting and guiding movement in a testing setup. The CRS 708 ensures controlled, repeatable motion for consistent testing results. The CRS 708 may include rotation sensors to track the angular displacement and force feedback.

The testing system 700 includes a top plate 710 that acts as a support structure for the entire setup. The top plate 710 provides a stable mounting point for the pneumatic piston 702, the hip joint 704, and other components. The top plate 710 ensures rigidity and the stability of the testing system 700, preventing unwanted vibrations or shifts during testing.

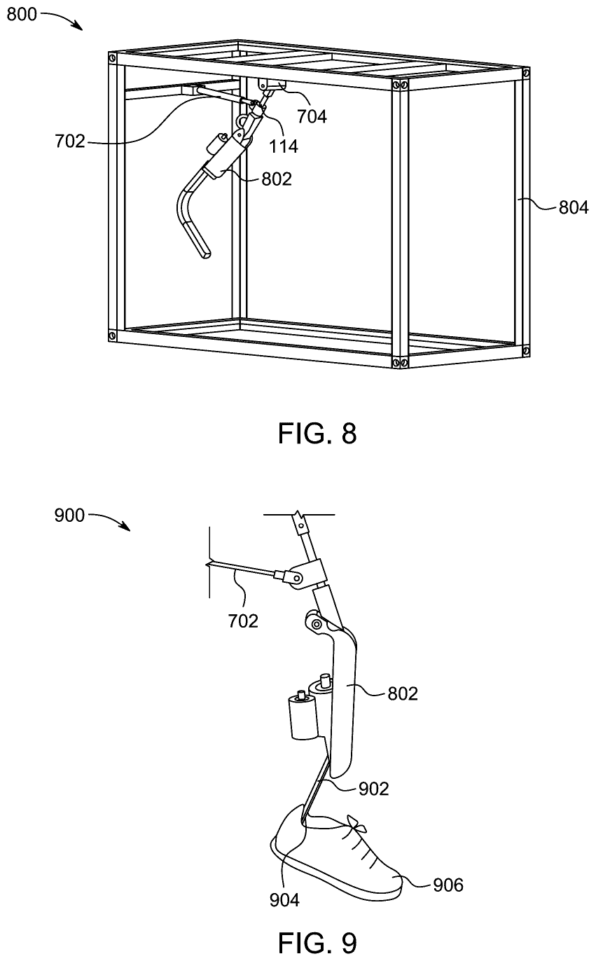

FIG. 8 illustrates a prosthetic limb testing system 800 using an Adaptive Prosthetic Knee (APK) 802 in a controlled environment, according to certain embodiments. The testing system 800 is designed to test and evaluate the movement, adaptability, and functionality of the prosthetic limb knee 106 under different conditions. The testing system 800 includes the pneumatic piston 702 that provides controlled motion to the prosthetic limb 102. The pneumatic piston 702 may be connected to the hip joint 704, providing the necessary force to move the femur 114 and the APK 802.

As discussed, the hip joint 704 acts as a pivot point, allowing the rotational movement similar to a natural human hip. The femur 114 represents the upper leg segment in the testing system 800. The femur 114 connects the hip joint 704 to the APK 802 and provides structural stability. The APK 802 represents an advanced knee joint that adapts to different movement conditions. The APK 802 includes the sensors, the actuators 222, and control algorithms to optimize knee movement based on the feedback. The APK 802 is tested for stability, responsiveness, and adaptability under different loading and movement conditions. The testing system 800 also includes aluminium extrusions 804 that form the structural framework of the prosthetic limb testing system 800. The aluminium extrusions 804 may provide a rigid and stable support for all mounted components. The aluminium extrusions 804 may be lightweight and durable and allow easy adjustments for experimental conditions.

FIG. 9 illustrates a schematic representation of prosthetic limb prototype 900, according to certain embodiments. The prosthetic limb prototype 900 serves as a robotic or a biomechanical testing setup designed to evaluate the functionality and performance of the APK 802. The prosthetic limb prototype 900 includes metallic components, such as the APK 802, the pneumatic piston 702, a pylon 902, and a prosthetic foot 904 housed in a shoe-like structure 906. The APK 802 is a cylindrical shaped component responsible for controlled knee flexion and extension, ensuring smooth and adaptive movement. The pneumatic piston 702 may be connected to the hip joint (not shown), providing the necessary force to move the femur (not shown) and the APK 802, allowing for natural motion. Below the knee joint, the pylon 902 acts as a structural connector between the knee and foot. The pylon 902 may be made from lightweight and durable materials such as aluminum or carbon fiber. At a base, the prosthetic foot 904, enclosed in the shoe-like structure 906, ensures proper ground contact, weight distribution, and stability. The entire prototype 900 is mounted on a rigid testing frame, which is used in a biomechanical lab setting to evaluate the gait patterns, the joint forces, and actuator performance under simulated walking conditions. In an embodiment, a setup includes wires and sensors connected to the prosthetic limb 102, indicating a presence of electronic control mechanisms for real-time feedback and performance monitoring.

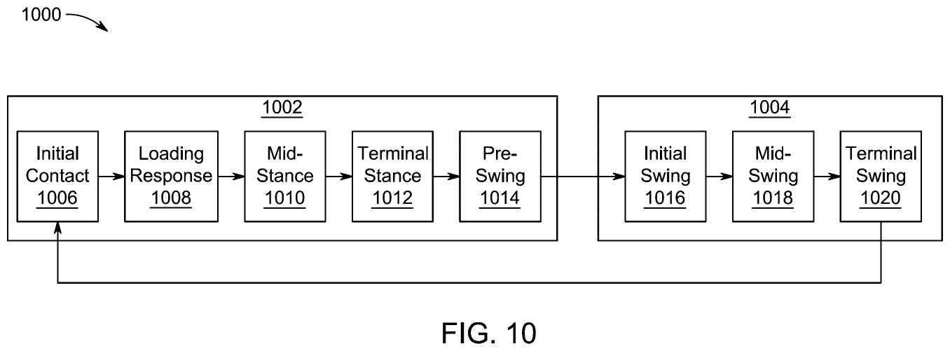

FIG. 10 illustrates a visualization of prosthetic control adaptation 1000, according to certain embodiments. The visualization of the prosthetic control adaptation 1000 represents how the prosthetic limb 102 closely mimics the natural gait cycle by dynamically adjusting the prosthetic outputs 236 at each phase of walking. In an embodiment, the natural gait cycle may be divided into phases such as the stance phase 1002 and the swing phase 1004. The stance phase 1002 includes 60% of the natural gait cycle, and the swing phase 1004 includes 40% of the natural gait cycle. Further, the stance phase 1002 includes various sub-phases such as an initial contact 1006 (i.e., a moment the foot touches the ground), the loading response 1008 (i.e., a period right after the initial contact 1006 where the body begins to bear weight on a leading leg), the mid-stance 1010 (i.e., when the body is directly over a weight-bearing foot), the terminal stance 1012 (when the heel of the weight-bearing foot begins to lift off the ground) and the pre-swing 1014 (a final phase of stance where the foot prepares to leave the ground).

Further, the swing phase 1004 includes sub-phases such as an initial swing 1016 (i.e., a period when the foot has just left the ground), a mid-swing 1018 (i.e., a phase where the leg moves forward as the knee begins to extend) and a terminal swing 1020 (a final phase just before the foot makes contact with the ground again).

Referring to FIG. 10, at each phase of the human gait cycle, the prosthetic limb 102 dynamically adjusts the response based on the sensor data. During the initial contact 1006, the pressure sensors 208 detect an initial ground contact. The control system 218 rapidly adjusts the damping to cushion the impact, mimicking the natural shock absorption of the unaffected leg 110. During the loading response 1008, as the body weight shifts onto the prosthetic limb 102, the control system 218 dynamically increases the support to ensure the stability and to prevent excessive knee flexion. During the mid-stance 1010, the control system 218 adjusts the alignment and the stiffness to provide adequate support as the body moves over the prosthetic limb 102. During the terminal stance 1012 and the pre-swing 1014, the prosthetic limb 102 prepares for toe-off by adjusting joint resistance, ensuring smooth propulsion at the end of the stance phase 1002.

During the initial swing 1016, the control system 218 modulates the actuators 222 of the knee and the hip to initiate forward motion, ensuring that the prosthetic limb 102 moves like the unaffected leg 110 while preventing excessive knee flexion. During the mid-swing 1018, the sensors monitor the position of the prosthetic limb 102, and the control system 218 modulates the movement of the prosthetic limb 102 to ensure a smooth leg swing and prepares for a next ground contact. During the terminal swing 1020, the control system 218 fine-tunes the damping and prepares for impact by adjusting resistance, ensuring smooth and stable foot placement for the next gait cycle.

The control system 218 of the prosthetic limb 102 continuously processes the sensor data to replicate the biomechanics of the unaffected leg 110, ensuring smooth transitions between the stance phase 1002 and the swing phase 1004 for stability and efficiency.

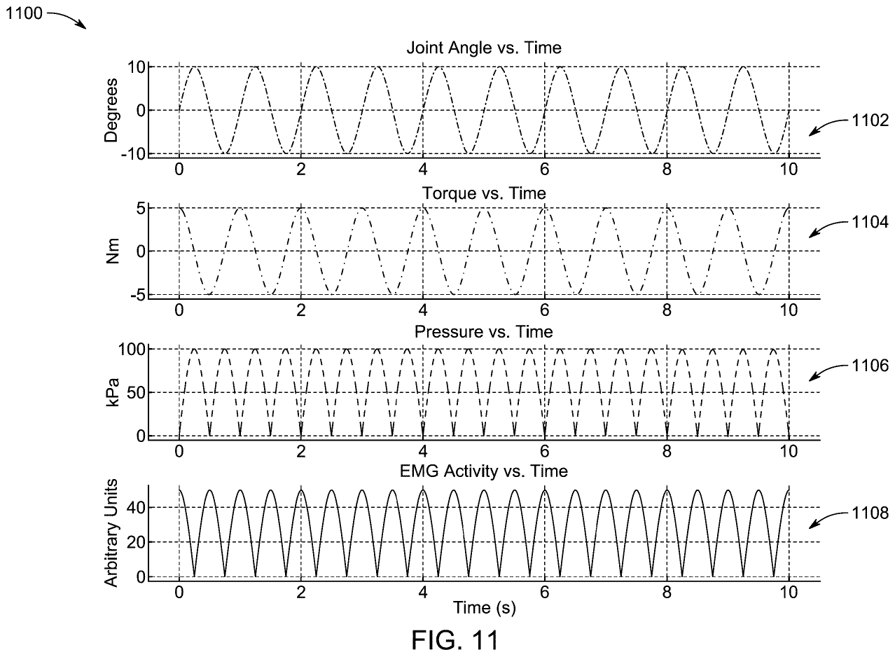

FIG. 11 illustrates a visual representation of simulated data 1100 collected over a predefined period, according to certain embodiments. In a preferred embodiment, the predefined period may be 10-second period (showing 10 gait cycles). Here, a scenario is simulated where the prosthetic outputs 236, such as the joint angles, the torque, the pressure and sensory feedback is plotted over a course of the gait cycle to capture the full dynamics of the gait cycle. The joint angles (degrees) reflect how the knee angle changes during the gait cycle. The torque (Newton-meters (Nm)) shows the torque applied by the prosthetic limb knee 106 to assist or resist the movement. The pressure data (Kilopascals (kPa)) from the pressure sensors 208 indicate the ground contact and the pressure distribution. The electrical activity data (having arbitrary units) reflects the muscle activity, which is helpful during the swing phase 1004 to monitor muscle engagement and predict the limb movement. The simulation assumes that the data is collected from the sensors embedded in the prosthetic limb 102 during a controlled walking test on the treadmill 706, which includes varying speeds and inclines to simulate different walking scenarios.

In an embodiment, time-series plots may be created for the complete gait cycle, segmented into the stance phase 1002 and the swing phase 1004. Each time-series plot provides a clear visual of how these data oscillate over the duration of 10 gait cycles. The time-series plots may represent joint angle vs. time plot 1102, torque vs. time plot 1104, pressure vs. time plot 1106, and electrical activity vs. time plot 1108. The joint angle vs. time plot 1102 represents a sinusoidal variation of the joint angles, reflecting a smooth transition of the knee angle from the initial contact 1006 (slight flexion) to the mid-stance 1010 (peak extension) and back to flexion for swing initiation. This pattern repeats every second for 10 cycles.

The torque vs. time plot 1104 follows a cosine waveform (consistent over 10 seconds), representing the torque applied by the prosthetic limb knee 106. The torque is minimal during the mid-stance 1010 (where gravity aids in leg extension) and increases during the transition to the swing phase 1004 to assist in lifting the leg.

The pressure vs. time plot 1106 represents an absolute value of a sinusoidal function, illustrating pressure variations across the gait cycles. The pressure vs. time plot 1106 also shows peaks at the initial contact 1006 and the toe-off, with sinusoidal variations throughout the gait cycle, indicating how the pressure shifts through the foot.

The electrical activity vs. time plot 1108 depicts the active muscle engagement as an absolute value of a cosine function, with elevated activity during the initial swing 1016.

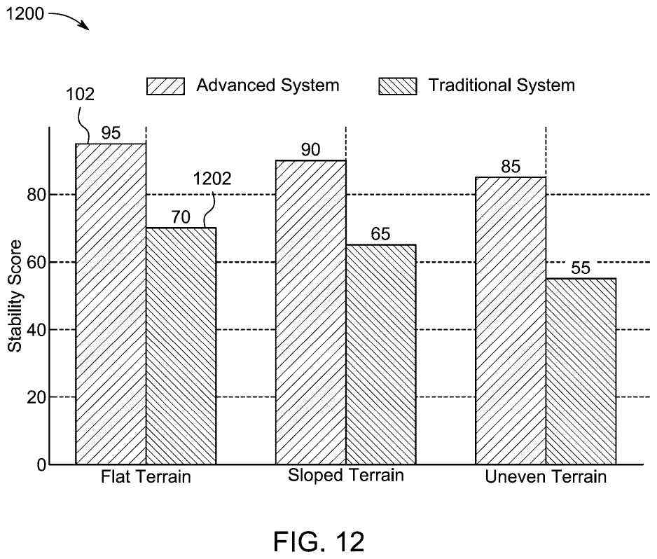

FIG. 12 illustrates a graph 1200 representing stability on varied terrain, according to certain embodiments. The graph 1200 represents a comparison of stability performance of the prosthetic limb 102 with traditional prosthetic systems across the different types of terrains such as the flat terrain, the slope terrain, and the uneven terrain. Here, X-axis of the graph 1200 represents the type of terrain, with the flat terrain representing a smooth and level surface, the slope terrain representing an inclined or sloped surface, and the uneven terrain representing a rugged and irregular surface with the obstacles. Y-axis measures a stability score, where α higher score indicates better stability, with factors like balance maintenance, posture control, and resistance to stability contributing to this score.

Further, the graph 1200 features two sets of bars for each terrain type. A bar with left slanted vertical lines represents the proposed prosthetic limb 102 and a bar with right slanted vertical lines represents the traditional prosthetic limb 1202. The length of each bar corresponds to the stability score achieved by the proposed prosthetic limb 102 and the traditional prosthetic limb 1202 on the respective terrain. On the flat terrain, the proposed prosthetic limb 102 and the traditional prosthetic limb 1202 show similar stability, though the proposed prosthetic limb 102 may show slight improvements due to its adaptive features like fine-tuned stiffness and damping adjustments. On the slope terrain, the proposed prosthetic limb 102 outperforms the traditional prosthetic limb 1202 with a higher stability score. The proposed prosthetic limb 102 may adjust the stiffness and the damping dynamically, which enhances the subject's ability to walk uphill or downhill without losing balance. Similarly, on the uneven terrain, the proposed prosthetic limb 102 exhibits much better stability, with its ability to adjust the joint angles, the damping and the stiffness in real-time, helping the users to maintain the balance on the rough surfaces. The graph 1200 visually demonstrates that the proposed prosthetic limb 102 outperforms the traditional prosthetic limb 1202 in terms of the stability across the different terrains.

In an embodiment, a graph (i.e., line graph) (not shown) may be plotted to illustrate the energy efficiency of the proposed prosthetic limb 102 compared to the traditional prosthetic limb 1202. In this graph, the X-axis represents a time during activity (in minutes), and Y-axis shows the energy consumed (in Watt-hours). The graph shows that the proposed prosthetic limb 102 consumes significantly less energy over time than the traditional prosthetic limb 1202. The lower energy consumption is due to optimized power management strategies of the proposed prosthetic limb 102, which ensures efficient use of battery resources. The result is extended battery life, offering the users more time between charges and reducing the overall cost of operation.

In an embodiment, a graph (not shown) may be plotted to illustrate user satisfaction ratings based on surveys conducted with the users of both the proposed prosthetic limb 102 and the traditional prosthetic limb 1202. In an embodiment, two pie charts may be presented, one for the proposed prosthetic limb 102 and another for the traditional prosthetic limb 1202, with slices representing percentages of the users who reported different satisfaction levels: very satisfied, satisfied, neutral, and dissatisfied. The proposed prosthetic limb 102 shows a higher percentage of the users reporting being very satisfied or satisfied, reflecting its superior comfort, functionality, and performance. The greater satisfaction is a direct result of the improved adaptability, stability, and energy efficiency of the proposed prosthetic limb 102.

Further, in an alternative embodiment, a graph (i.e., line graph) (not shown) may be plotted for comparing a response time of the proposed prosthetic limb 102 and the traditional prosthetic limb 1202, showing how quickly the proposed prosthetic limb 102 and the traditional prosthetic limb 1202 adjusts to sudden changes in the terrain or the speed. In this graph, X-axis represents instances of the terrain or speed changes, while Y-axis tracks the response time (in seconds). The proposed prosthetic limb 102 outperforms the traditional prosthetic limb 1202, with significantly faster response times. This enhanced adaptability ensures that the users easily navigate varying terrains, transitioning smoothly from one type of surface to another without compromising stability or comfort. The quick adjustments made by the proposed prosthetic limb 102 are especially beneficial for the users engaged in dynamic activities, such as walking on the uneven surfaces or changing their walking speed rapidly.

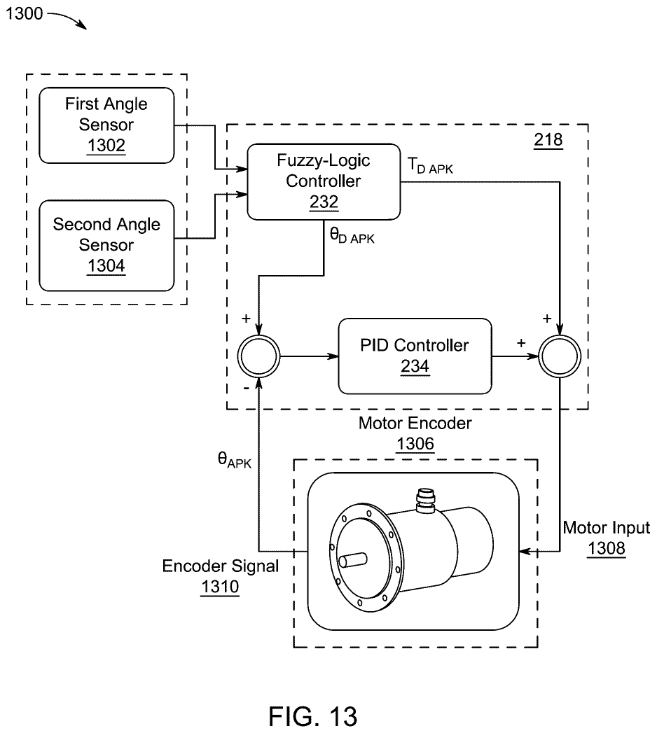

FIG. 13 illustrates a hybrid Fuzzy-PID control architecture 1300 (hereinafter referred to as the architecture 1300), according to certain embodiments. The architecture 1300 may be designed to optimize the movement and stability of the APK 802. In an embodiment, the control system 218 integrates inputs from the multiple sensors and utilizes both predictive (feed-forward) and corrective (feedback) mechanisms to ensure smooth and responsive prosthetic control.

The architecture 1300 includes a first angle sensor 1302 and a second angle sensor 1304. The first angle sensor 1302 and the second angle sensor 1304 may be attached to the unaffected leg 110 of the subject 100. In a preferred embodiment, the first angle sensor 1302 and the second angle sensor 1304 may be the accelerometer. The first angle sensor 1302 and the second angle sensor 1304 may be configured to measure the joint angles at critical points (e.g., hip, knee, or ankle). The first angle sensor 1302 and the second angle sensor 1304 may provide real-time positional data as the inputs to the fuzzy logic controller 232 (i.e., feed-forward design), enabling the control system 218 to predict and initiate appropriate knee movements.

The fuzzy logic controller 232 receives the inputs from the first angle sensor 1302 and the second angle sensor 1304 and processes the inputs to generate the control signal based on predefined rules and empirical data (standard data collected from real-world observations). In other words, the fuzzy logic controller 232 predicts the required movements of the prosthetic limb knee 106 without waiting for the feedback, allowing quick responses under normal and expected conditions. In an exemplary embodiment, a desired torque for the APK 802 (TD APK) is the control signal representing a torque demand calculated by the fuzzy logic controller 232, which is sent directly to the motor encoder 1306 to control a rotational force of the prosthetic limb knee 106. Similarly, a desired angle for the APK 802 (OD APK) represents a desired angular position, that may be sent to the PID controller 234 to assist in fine-tuning the knee movement.

The fuzzy logic controller 232 is effective in stable environments, and less reliable under unpredictable conditions, such as the uneven terrain or sudden changes in the walking speed. Therefore, the fuzzy logic controller 232 lacks real-time correction mechanisms to respond to unexpected disturbances. To overcome the limitations of the fuzzy logic controller 232 (i.e., feed-forward approach), a feedback loop is integrated into the control system 218. The feedback loop includes the PID controller 234, which continuously monitors the real-time position of the APK 802. In an embodiment, the PID controller 234 calculates an error signal, which represents the difference between the desired knee position (OD APK) and an actual position of the APK 802. The error signal enables the control system 218 to make precise adjustments to the knee movement. In other words, the PID controller 234 utilizes the error signal to adjust a motor input 1308 for precise control of the APK 802. The motor encoder 1306 receives the motor input 1308 from both the fuzzy logic controller 232 and the PID controller 234. The motor encoder 1306 tracks the motor's performance and outputs an encoder signal 1310 back to the PID controller 234, ensuring continuous feedback and adjustment. In an exemplary embodiment, the motor encoder 1306 provides the real-time feedback, which the PID controller 234 uses to correct deviations, ensuring the prosthetic limb knee 106 operates smoothly even under varying conditions. The architecture 1300 enables the prosthetic limb knee 106 to respond dynamically to both predictable and unpredictable situations, providing a more natural and stable gait for the user.

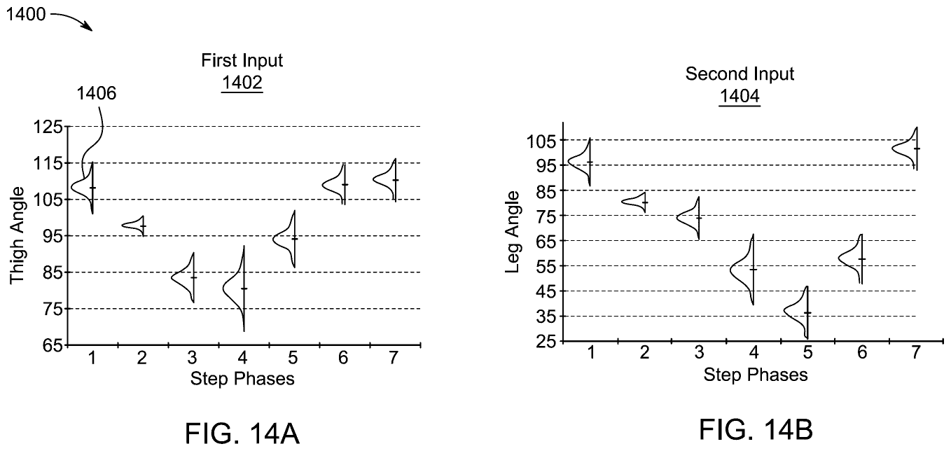

FIGS. 14A and 14B illustrate the input membership functions 1400 of the fuzzy logic controller 232, according to certain embodiments. The input membership functions 1400 are configured to map the angular movement data to the specific gait cycle phases. The input membership functions 1400 classify the thigh and the leg angles of the unaffected leg 110 into fuzzy sets, allowing a rule-based Fuzzy Inference System (FIS) to determine the most likely gait phase of the APK 802.

The FIS operates by analyzing real-time angular movement data from two primary inputs such as a first input 1402, which corresponds to the thigh angle measured by the first angle sensor 1302, and the second input 1404, which corresponds to the leg angle measured by the second angle sensor 1304. The first input 1402 and the second input 1404 provide critical data on how the unaffected leg 110 moves during walking, allowing the FIS to infer the appropriate gait phase for controlling the prosthetic limb 102. Based on the analysis, the fuzzy logic controller 232 generates a torque command for the motor encoder 1306, ensuring that the prosthetic limb 102 replicates natural movement dynamics in a controlled and adaptive manner.

As depicted in FIG. 14A, the thigh angle varies across different step phases, while FIG. 14B shows the corresponding leg angle variations. These figures use a distribution plot 1406 to illustrate how angular movement transitions across different gait phases, emphasizing the evolving dynamics of the prosthetic limb 102 over time. The FIS within the fuzzy logic controller 232 processes the angular movement data by first converting values of the angular movement data into fuzzy variables such as a small knee flexion, a moderate knee flexion, and a high knee flexion, allowing for a smooth classification of movement states.

Once the angular movement data is classified, the FIS applies predefined fuzzy rules to determine the appropriate adjustment. For example, if the thigh angle increases while the leg angle remains moderate, then the FIS increases the knee damping to maintain the stability. Conversely, if the leg moves rapidly while the thigh angle is large, the FIS applies higher resistance to prevent abrupt motion. By continuously analyzing angular variations of the thigh and the leg through the distribution plot 1406, the fuzzy logic controller 232 ensures a natural and adaptive gait cycle, enhancing both mobility and stability for the user.

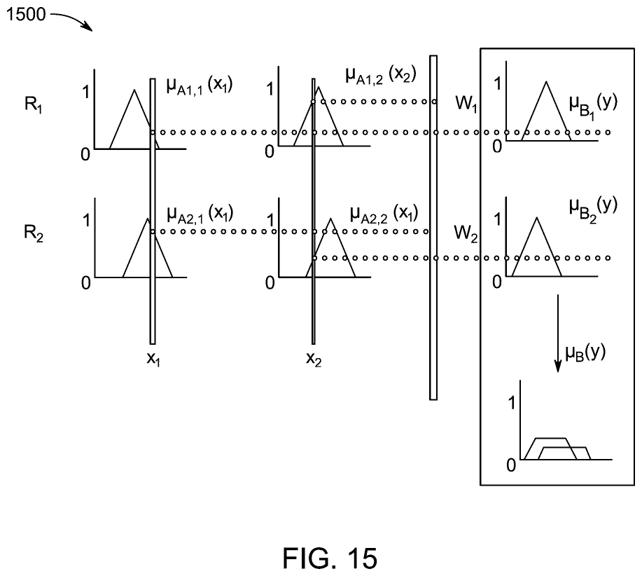

FIG. 15 illustrates a fuzzy inference process 1500 used in the fuzzy logic controller 232, according to certain embodiments. The fuzzy inference process 1500 processes the angular movement data from the unaffected leg 110 to determine appropriate adjustments for the prosthetic limb 102. The fuzzy inference process 1500 includes three primary stages, such as fuzzification, rule evaluation, and defuzzification, ensuring that the prosthetic limb 102 follows the natural and adaptive gait pattern. As used herein, the term “fuzzification” refers to converting crisp numerical inputs into fuzzy values (linguistic variables) that may be processed by the FIS. As used herein, the term “defuzzification” refers to a process of converting a fuzzy output set into a single value that may be used as a precise control action or decision.

The rule-based FIS is structured into primary components such as the input membership functions 1400, which translate the angular movement data into the specific gait cycle phases, and a set of “if-then” rules that emulate human reasoning. In an embodiment, inputs to the fuzzy logic controller 232 include thigh (x₁) and leg (x₂) angles, measured from the first angle sensor 1302 and the second angle sensor 1304 of the unaffected leg 110. The inputs pass through the input membership functions (UA) 1400, which classify the thigh and leg angles into fuzzy sets such as low, medium, and high. The input membership functions 1400 may be represented in a triangular-shaped plot, defining a degree to which an input value belongs to a particular fuzzy set.

In an exemplary embodiment, the input membership function 1400 for each input is given by μ_{Ai,j} (xⱼ) where A_{i,j} refers to the fuzzy set for xⱼ, i represents a rule index (e.g., R₁, R₂) and j represents an input variable index (1 for x₁ and 2 for x₂). Each rule (R₁, R₂, etc.) has an associated membership function for x₁ and x₂, determining how much the given input value belongs to each fuzzy set.

Each gait phase, such as the initial contact 1006, the loading response 1008, the mid-stance 1010, the terminal stance 1012, the pre-swing 1014, the initial swing 1016, the mid-swing 1018 and the terminal swing 1020, has a corresponding membership function associated with the thigh angle and the leg angle of the unaffected leg 110. Once the gait phase is identified, a firing of specific rules within the rule-based FIS activates associated output membership functions, ensuring smooth transitions between the gait phases.

The fuzzy logic controller 232 applies fuzzy rules to determine the gait phase (y) based on the inputs x₁ (thigh angle) and x₂ (leg angle). The fuzzy rules follow an “if-then” structure, mapping the input variables to an output gait phase. Each rule follows a structure of:

IF x₁ is A_{i,1} AND x₂ is A_{i,2} THEN y is Bᵢ

Example of the fuzzy rules include:
IF x₁ is LR AND x₂ is LR THEN y is LR (loading response 1008)
IF x₁ is MST AND x₂ is MST THEN y is MST (mid-stance 1010)
IF x₁ is TST AND x₂ is TST THEN y is TST (terminal stance 1012)
IF x₁ is PSW AND x₂ is PSW THEN y is PSW (pre-swing 1014)
IF x₁ is ISW AND x₂ is ISW THEN y is ISW (initial swing 1016)
IF x₁ is MSW AND x₂ is MSW THEN y is MSW (mid-swing 1018)
IF x₁ is TSW AND x₂ is TSW THEN y is TSW (terminal swing 1020)

For each rule Rᵢ, a degree of activation (firing strength, Wᵢ) is determined by combining input membership values of x₁ and x₂. The firing strength Wᵢ for each rule Rᵢ is computed using equation (9):

$${W}_{i} = {μ}_{{A}_{i , 1}} ( {x}_{1} ) \cdot {μ}_{{A}_{i , 2}} ( {x}_{2} ) , \qquad ( 9 )$$

where Wᵢ represents a strength of rule activation based on the input values. The product of the two input membership functions (i.e., μ_{Ai,1}·μ_{Ai,2}) 1400 represents an intersection of both conditions in the fuzzy rule. A higher firing strength Wᵢ indicates a stronger rule activation, meaning it has a more significant influence on a final control output. W₁ and W₂ represent the firing strengths of two different rules (R₁ and R₂), which may be used to determine the final gait phase (y).

After rule evaluation, the FIS computes the output membership functions (μ_{B}), determining the final control outputs (torque command for the prosthetic limb 102). The output membership function (μ_{B}) for each rule is denoted as μ_{Bi} (y), where Bᵢ represents an output fuzzy set for rule Rᵢ, and yᵢ represents the final control outputs (torque command). The final control outputs are functions of x₁ and x₂, computed using equations (10) and (11):

$${y}_{1} = {f}_{1} ( {x}_{1} , {x}_{2} ) , \qquad ( 10 )$$

$${y}_{2} = {f}_{2} ( {x}_{1} , {x}_{2} ) , \qquad ( 11 )$$

The equations (10) and (11) indicate that the final control outputs y₁ and y₂ are functions of the input angular readings from x₁₂ and x₂. To obtain a precise control action, the FIS performs the defuzzification, converting the fuzzy outputs (final control outputs) into a single crisp value, which determines the appropriate torque command. The final control output (y) is computed using a weighted average method:

$$y = \frac{{w}_{1} {f}_{1} + {w}_{2} {f}_{2}}{{w}_{1} + {w}_{2}} , \qquad ( 12 )$$

where y represents the final control output (i.e., final control signal), and w₁ and w₂ are the firing strengths of the rules R₁ and R₂, respectively. The equation (12) ensures that the fuzzy rules with higher activation weights contribute more to a final decision, leading to a smooth and precise torque command. In an embodiment, the FIS may use a Center of Heights (CoH) technique for defuzzification, which further refines the final control output for accurate motion control.

To improve the fuzzy logic controller 232, an adaptive tuning method may be applied using an Adaptive Network-Based Fuzzy Inference System (ANFIS). The ANFIS framework fine-tunes the input membership functions 1400 and the rules to enhance accuracy. The tuning process involves adjusting parameters such as the mean and standard deviation of Gaussian-based membership functions, similar to training an artificial neural network. The Gaussian functions are advantageous due to its smooth and symmetric properties, allowing for efficient calculations while closely approximating natural limb movement dynamics. By incorporating probabilistic reasoning, a decision-making under uncertainty may be enhanced, making it robust and adaptable to user-specific gait variations.

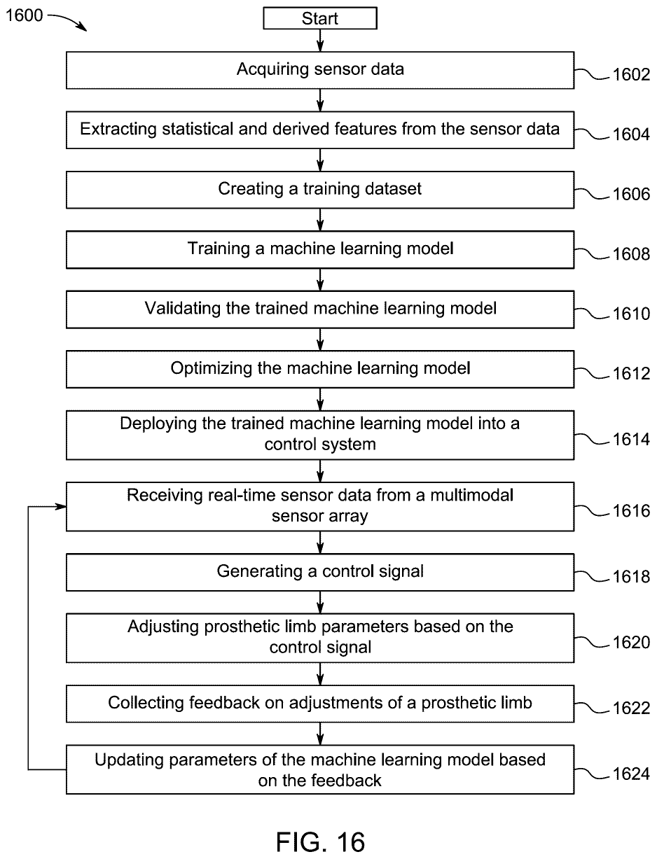

FIG. 16 illustrates a flowchart of a method 1600 for training the machine learning model 230, according to certain embodiments.

At step 1602, the method 1600 includes acquiring the data, such as the sensor data, the subject intent, and the prosthetic limb parameters through the multimodal sensor array 202 and biomechanical analysis tools (i.e., inertial measurement units, gait analysis treadmills, and so forth). In an embodiment, the sensor data may be collected from the sensors embedded within the prosthetic limb 102, including the inclinometers 212, the gyroscopes 206, the ultrasonic sensors 214, the pressure sensors 208, and so forth. The sensors capture real-time environmental and biomechanical data, such as the inclination angles, the angular velocity, the pressure distribution, the ground contact forces, and the obstacle proximity. The sensor data may be utilized to understand a current movement state and environmental interactions of the prosthetic limb 102.

In an embodiment, the sensor data may be collected from the sensors attached on the unaffected leg 110, including the first angle sensor 1302 (thigh angle) and the second angle sensor 1304 (leg angle). The sensors track the natural gait cycle and provide a reference for movement intent, allowing the prosthetic limb 102 to synchronize its adjustments based on user's expected motion.

To build an effective training dataset, corresponding successful adjustment parameters such as the stiffness, the damping coefficients, and the flexion angles may be recorded alongside the sensor readings. The parameters may be captured through actuator feedback and encoder measurements, ensuring that the training dataset contains optimal knee adjustments applied during various walking conditions (e.g., flat surfaces, inclines, stairs, and obstacle navigation).

In an embodiment, the collected data is structured into input-output pairs, where the sensor readings from both the unaffected leg 110 and the prosthetic limb 102 serve as inputs, and the optimal adjustment parameters serve as outputs. This structured dataset allows the machine learning model 230 to learn how to predict the most effective knee adjustments based on real-time sensor data, ensuring adaptive and precise prosthetic control.

At step 1604, the method 1600 includes extracting the features from the collected sensor data. This step includes preprocessing the collected sensor data, where raw sensor signals are filtered to remove the noise using the signal processing techniques. This step further includes extracting statistical features, including mean, median and variance. The mean represents an average sensor reading over a specific time window, the median indicates a central tendency, reducing an effect of outliers, the variance measures a variability or dispersion of the sensor readings, reflecting fluctuations in the gait dynamics or the environmental changes.

In addition to the statistical features, this step further includes calculating derived features (i.e., change over time and signal gradient) to capture dynamic aspects of the movement. For instance, the change over time is determined by computing a difference between consecutive sensor readings, emphasizing sudden shifts in the angles or the forces. The signal gradient measures the rate of change of the sensor data, indicating acceleration or deceleration trends, which may be essential for detecting rapid movements or adjustments in the walking patterns. The statistical and derived features collectively enhance the ability of the machine learning model 230 to predict optimal prosthetic adjustments under varying conditions.

At step 1606, the method 1600 includes creating the training dataset by structuring the extracted features into the input-output pairs for training the machine learning model 230. The training dataset may be prepared to map the sensor data and the subject intent to the optimal prosthetic limb adjustments, ensuring adaptive and precise knee control.

In an embodiment, the input data includes pre-processed and feature-extracted sensor data from both the prosthetic limb 102 and the unaffected leg 110. This includes kinematic features (e.g., joint angles, angular velocity, acceleration), environmental features (e.g., terrain inclination, obstacle proximity), and subject intent indicators (e.g., muscle activity from the EMG sensors 210). The output data includes the prosthetic limb parameters representing the optimal adjustments required for different walking scenarios. The output data is recorded from actuator feedback and encoder measurements, ensuring that the training data captures real-world mechanical responses.

At step 1608, the method 1600 includes training the machine learning model 230 by feeding the prepared training dataset into a regression-based machine learning model such as random forest, gradient boosting regression, and so forth. The machine learning model 230 learns a mapping function between the sensor features (e.g., inclination angles, gait pressure, signal gradients) and the corresponding prosthetic limb adjustments (e.g., knee stiffness, damping coefficients). During training, the machine learning model 230 minimizes a loss function such as a Mean Squared Error (MSE), adjusting its internal parameters to better predict adjustments for various walking scenarios. In an embodiment, the training may be conducted using a batch processing approach, where the training dataset is divided into smaller portions, allowing the machine learning model 230 to iteratively refine the predictions. In an embodiment, the trained machine learning model 230 captures complex relationships between gait mechanics, terrain changes, and necessary prosthetic outputs 236.

At step 1610, the method 1600 includes validating the trained machine learning model 230 to assess its generalization ability. In an embodiment, validating the trained machine learning model 230 includes fine-tuning hyperparameters by evaluating the performance of the machine learning model 230 on unseen data. In an embodiment, cross-validation techniques such as k-fold cross-validation may be used to prevent overfitting by training the machine learning model 230 on different subsets of data while ensuring performance consistency. Further, validation metrics, including Mean Absolute Error (MAE), Root Mean Squared Error (RMSE), and R₂ score may be calculated to determine how accurately the machine learning model 230 predicts the prosthetic adjustments across varying terrains and movements. If the machine learning model 230 underperforms, then adjustments in the hyperparameters may be performed before deployment of the machine learning model 230.

At step 1612, the method 1600 includes optimizing the machine learning model 230 using optimization techniques to enhance the performance. This step ensures that the machine learning model 230 achieves higher accuracy, efficiency, and adaptability in real-world gait conditions. In an embodiment, optimizing the machine learning model 230 includes tuning the hyperparameters by adjusting parameters of the machine learning model 230. The parameters may be a learning rate, number of hidden layers (for neural networks), a decision tree depth (for ensemble models), and regularization factors. This step further includes determining optimal set of hyperparameters by using various techniques such as grid search, random search, and bayesian optimization, and so forth that minimize prediction errors and improve model generalization across diverse walking condition.

At step 1614, the method 1600 includes deploying the trained machine learning model 230 into the control system 218, ensuring seamless operation in real-world conditions. This step includes embedding the trained machine learning model 230 onto an edge computing hardware, such as the microcontroller or the FPGA, within the prosthetic limb 102. This step enables low-latency decision-making, ensuring rapid response to the user movements. The machine learning model 230 may be programmed to interact with the sensor data, the data processing unit 216, and the actuators 222, enabling real-time prosthetic adjustments. Before deployment of the machine learning model 230, a Hardware-In-The-Loop (HIL) testing may be performed to verify the compatibility of the machine learning model 230 with embedded systems and ensure robustness in various walking conditions.

At step 1616, the method 1600 includes continuously receiving real-time sensor data, by the data processing unit 216, from the multimodal sensor array 202. The sensor data may be fed into the trained machine learning model 230 for real-time decision-making.

At step 1618, the method 1600 includes generating, by the control system 218, the control signal having the control parameters for adjusting the movement of the prosthetic limb 102. The control parameters include the values of the prosthetic limb parameters to be adjusted.

At step 1620, the method 1600 includes physically adjusting, by the actuators 222, the prosthetic limb 102 by adjusting the prosthetic limb parameters based on the control parameters.

At step 1622, the method 1600 includes collecting feedback on the prosthetic limb's adjustments by continuously tracking the performance metrics such as the joint angles, the gait symmetry, the movement speed, and the stability.

At step 1624, the method 1600 includes updating the parameters of the machine learning model 230 based on the feedback collected in step 1622. This step involves refining the machine learning model 230 to improve the ability of the machine learning model 230 to predict optimal prosthetic adjustments by incorporating new performance data and user feedback. This step further includes retraining or fine-tuning the machine learning model 230 to enhance the accuracy in different gait conditions. In an embodiment, adjustments may be made to sensor thresholds and feature weightings, ensuring the machine learning model 230 adapts better to the user movement patterns and the environmental changes for more precise and responsive knee control.

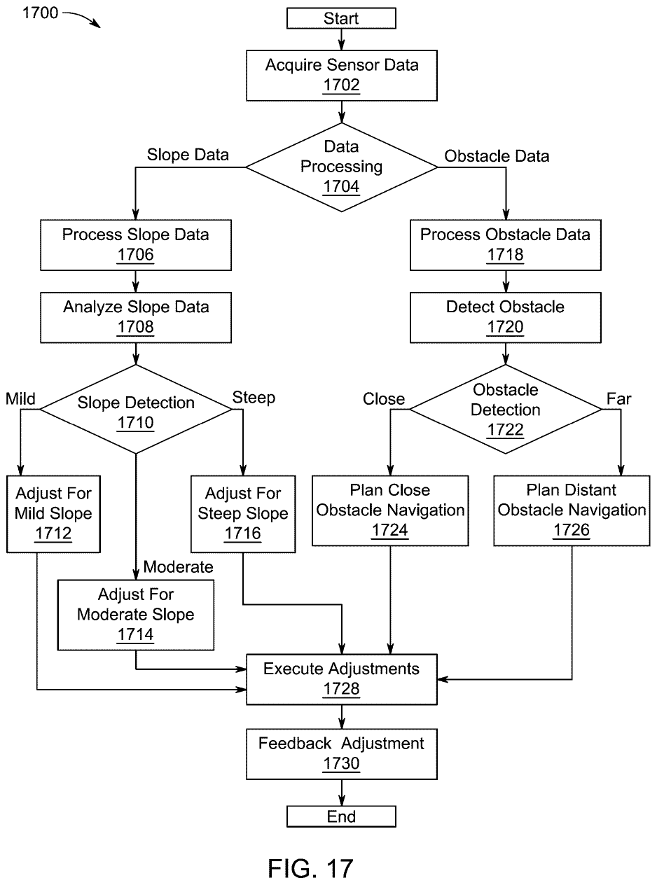

FIG. 17 illustrates a flowchart of a method 1700 for enabling the slope navigation and the obstacle detection in the prosthetic limb 102, according to certain embodiments. The method 1700 dynamically adjusts the prosthetic limb parameters based on the real-time sensor data to enhance the mobility, balance and safety.

At step 1702, the method 1700 includes acquiring the sensor data from the multimodal sensor array 202. In an exemplary embodiment, the sensor data may be acquired from the inclinometers 212, the gyroscopes 206 and the ultrasonic sensors 214. This step includes obtaining the inclination angle data for a specified period from the inclinometer 212. In an exemplary embodiment, the inclinometer 212 is a device that measures the angular tilt of the prosthetic limb 102 relative to a horizontal plane (ground level). In such embodiment, the inclinometer 212 detects the inclination angle in degrees and helps determine whether the prosthetic limb 102 is walking on the flat surface, incline or decline. The inclinometer 212 may include a Micro-Electro-Mechanical System (MEMS) accelerometer that measures gravitational acceleration. For example, the inclinometer 212 reads 0° for the flat surface, reads 5° for a mild slope and greater than 15° for a steep slope.

This step also includes obtaining the angular motion data (useful for differentiating between intentional changes in posture (user-initiated movements) and actual slope changes) for the specified period from the gyroscopes 206. For example, if both the inclinometer 212 and the gyroscope 206 detect the inclination without the body movement, then it indicates an actual slope, and if the inclinometer 212 detects the inclination but the gyroscope 206 shows rapid motion, then it indicates the user-initiated posture change (e.g., leaning forward). In an exemplary embodiment, the gyroscope 206 contains a vibrating MEMS structure that detects changes in an angular momentum when the prosthetic limb 102 rotates. The gyroscope 206 measures rotation rates in degrees per second along different axes. This step also includes obtaining the distance data to potential obstacles in the path of the subject 100 from the ultrasonic sensors 214. In an exemplary embodiment, the ultrasonic sensors 214 may measure the distance to nearby obstacles by emitting sound waves and calculate a time the sound waves take to reflect back. In an embodiment, obtaining the distance data includes filtering the distance data using a filtering technique (e.g., moving average filter) to stabilize readings of the distance data. The filtering technique reduces the impact of sudden fluctuations or the noise that is present in the distance data due to environmental factors such as sensor interference, rapid user movements, and so forth. In an exemplary embodiment, the moving average filter works by continuously calculating an average of a set of most recent measurements of the distance data within a predefined time window (e.g., last 5-10 readings). As new data points are collected, oldest values are replaced, and the average is updated.

At step 1704, the method 1700 includes determining, by the data processing unit 216, whether the subject 100 is encountering the slope (i.e., uphill or downhill) or the obstacle (i.e., small step, large object, barrier, and so forth) based on sensor alerts or context. For example, if the inclinometer 212 detects a continuous upward lift, it indicates the slope, and if the inclinometer 212 detects a sharp tilt (15° in one step), it indicates a possible obstacle. Similarly, if the gyroscope 206 detects a smooth, slow change in the angle, it indicates a gradual slope. If the gyroscope 206 detects a sudden motion, it indicates that the subject 100 is actively stepping over something (i.e., the obstacle). If the ultrasonic sensor 214 detects an object 30 cm ahead, then it indicates the obstacle and if the ultrasonic sensor 214 detects no obstacle, but the inclinometer 212 shows a gradual incline, it is indicating the slope.

In an embodiment, if the method 1700 determines that the subject 100 is encountering the slope, the method 1700 proceeds to step 1706. In another embodiment, if the method 1700 determines that subject 100 is encountering the obstacle, then the method 1700 proceeds to step 1718.

At step 1706, the method 1700 includes processing, by the data processing unit 216, the inclination angle data to determine a degree of incline. This step ensures that the prosthetic limb 102 adapts correctly to different slopes by filtering, averaging and stabilizing the inclination angle data before making the adjustments. The processing includes applying the signal processing techniques such as the low pass filter to the inclination angle data to smooth out the noise and the fluctuations caused by sudden movements and environmental disturbances. The low-pass filter may allow low-frequency components (that represent gradual changes in the slope) to pass through while attenuating high-frequency noise. Upon filtering, the method 1700 includes calculating an average of recent inclination angle readings over a specified time window to determine steady-state angle. The steady-state angle reflects a stable measurement of the orientation of the prosthetic limb 102, minimizing the impact of temporary variations of short-term disturbances.

At step 1708, the method 1700 includes comparing, by the data processing unit 216, the inclination angle data (i.e., steady-state angle) with the threshold angle ranges to classify the slope of the path into a first category of multiple categories (such as mild, moderate, and steep).

At step 1710, the method 1700 includes determining, by the data processing unit 216, the adjustments to the prosthetic limb parameters based on the first category of the slope to generate adjusted prosthetic limb parameters. In an exemplary embodiment, the data processing unit 216 identifies the adjustments based on the first category of the slope.

At step 1712, the method 1700 includes performing slight knee-damping adjustments if the first category of the slope is the mild slope.

At step 1714, the method 1700 includes increasing knee stiffness for controlled descent or ascent if the first category of the slope is the moderate slope.

At step 1716, the method 1700 includes performing maximum stiffness and damping adjustments for stability if the first category of the slope is the steep slope.

At step 1718, the method 1700 includes processing, by the data processing unit 216, the distance data to derive obstacle parameters including a size, proximity, and a classification of the obstacle. This step includes identifying the potential obstacle based on the specified minimum safe distance. This step further includes comparing the distance data with the specified minimum safe distance to identify the proximity of the obstacle. The minimum safe distance may be set based on factors such as the walking speed of the subject 100, a reaction time of the prosthetic limb 102, and the range of motion required to avoid the obstacles. In an exemplary embodiment, if the distance data is greater than the minimum safe distance, then the proximity of the obstacle is considered as the far proximity. In another embodiment, if the distance data is less than or equal to the minimum safe distance, then the proximity of the obstacle is considered as the close proximity.

The method 1700 further includes determining the size of the potential obstacle. The size of potential obstacle may be small (e.g., <10 cm in height) or large (e.g., >10 cm). For example, the obstacle detected with the height of 8 cm and the width of 15 cm may be classified as small and the obstacle detected with the height of 35 cm is classified as large.

At step 1720, the method 1700 includes determining the classification (type) of the obstacle based on the size and the proximity of the obstacle. The type of obstacles may be bypassable obstacles (e.g., small debris, cracks in the pavement), step-over obstacles (e.g., low curbs, small rocks), step-side obstacles (e.g., large barriers, poles), complex obstacles (e.g., stairs, large uneven surfaces), and so forth.

At step 1722, the method 1700 includes determining, by the data processing unit 216, the adjustments (i.e., navigation strategy) to the prosthetic limb parameters based on the obstacle parameters to generate the adjusted prosthetic limb parameters. The adjustments ensure that the prosthetic limb 102 adapts appropriately to navigate the detected obstacle safely and efficiently. In an exemplary embodiment, the data processing unit 216 processes the obstacle parameters, such as the size and the proximity, to determine the necessary adjustments for seamless movement. In an embodiment, if the proximity of the obstacle is far and the size of the obstacle is small, the data processing unit 216 may trigger a stepping-over strategy. To facilitate this, the prosthetic knee flexion angle is increased during the swing phase 1004, allowing the foot to clear the obstacle without disrupting the gait cycle. Additionally, ankle dorsiflexion adjustments may be applied to ensure smooth foot placement upon landing. In another embodiment, if the proximity of the obstacle is close and the size of the obstacle is small, the data processing unit 216 may trigger an immediate step adjustment, increasing step height and modifying joint damping to enhance stability during obstacle clearance.

In yet another embodiment, if the proximity of the obstacle is far and the size of the obstacle is large, the data processing unit 216 may preemptively plan a side-step strategy, adjusting lateral stability parameters and modifying the joint stiffness to enable a smooth redirection of movement. For cases where the obstacle is large and close, the data processing unit 216 prioritizes the stability and controlled movement, potentially executing a full stop before determining whether the side-step or a more complex maneuver is required. This step also includes generating the control signal, by the control system 218, for obstacle avoidance based on the adjusted prosthetic limb parameters.

At step 1724, the method 1700 includes determining a navigation strategy (i.e., side-step or step over) for the close obstacles if the proximity of the obstacle is close and the size of the obstacle is either large or small.

At step 1726, the method 1700 includes determining the navigation strategy (i.e., side-step or step over) for the far obstacles if the proximity of the obstacle is far and the size of the obstacle is either large or small.

At step 1728, the method 1700 includes executing the necessary mechanical adjustments to the prosthetic limb 102 based on the decisions obtained from step 1710 (obstacle detection) and step 1722 (prosthetic adjustment determination). This involves transmitting the control signal to the actuator system 220, which adjusts the joint stiffness, the damping, and movement trajectory according to the determined navigation strategy.

At step 1730, the method 1700 includes monitoring outcomes through sensor feedback and adjusting the control parameters to optimize performance and comfort. This step involves continuously analyzing real-time data from the embedded sensors to assess the effectiveness of the executed adjustments. This step further includes comparing actual movement outcomes with the expected movement patterns, identifying any deviations or inefficiencies in obstacle negotiation. If discrepancies are detected, such as insufficient step height for stepping over the obstacle or instability during the side-step, the control system 218 dynamically modifies knee flexion, joint damping, and lateral stability parameters to improve performance. Additionally, the feedback from ground contact sensors ensures that post-movement stability is maintained, preventing unintended slips or balance issues.

The first embodiment is illustrated with respect to FIG. 1A-FIG. 17. The first embodiment discloses the prosthetic limb 102 for replacing a portion of a leg of a subject 100 is described. The prosthetic limb 102 includes a prosthetic limb body 104, a prosthetic limb knee 106 and a prosthetic limb ankle 108. The prosthetic limb 102 further includes a multimodal sensor array 202 embedded in the prosthetic limb body 104 to obtain sensor data. The sensor data are used to derive parameters that are indicative of (a) a physiological state of the subject 100 and (b) an environment in a proximity of the prosthetic limb 102. The prosthetic limb 102 further includes a data processing unit 216 that is configured to process the sensor data to generate a processed sensor data. The processed sensor data is generated using a machine learning (ML) model 230 trained on training data including sensor data and corresponding subject intent and corresponding prosthetic limb parameters. The ML model 230 outputting the processed sensor data as a predicted subject intent and predicted prosthetic limb parameters based on the sensor data. The prosthetic limb 102 further includes a control system 218 that is configured to generate a control signal having control parameters for adjusting a movement of the prosthetic limb 102. The control parameters include values of the prosthetic limb parameters to be adjusted. The control system 218 is configured to generate the control signal based on the processed sensor data and using a fuzzy logic controller 232 and a proportional-integral-derivative (PID) controller 234 of the control system 218.

In an aspect, the prosthetic limb 102 further includes an actuator 222 that is configured to physically adjust the prosthetic limb 102 by adjusting the prosthetic limb parameters based on the control parameters, wherein the prosthetic limb parameters include at least some of joint stiffness, joint angle, damping, motor speed or motor torque.

In an aspect, the prosthetic limb 102 further includes a haptic feedback system 240 to provide haptic feedback regarding positioning and movement of the prosthetic limb 102.

In an aspect, the sensor data includes at least one of: accelerometer data obtained using accelerometers 204a-204b mounted near the knee and the ankle of the prosthetic limb 102. The accelerometer data is used to derive a speed of the subject 100, a joint angle of joints of the prosthetic limb 102, both of which are indicative of a gait of the subject 100. The sensor data further includes angular motion data obtained using gyroscopes 206 mounted near the knee and ankle of the prosthetic limb 102. The angular motion data is indicative of angular orientation of one or more joints of the prosthetic limb 102, which is indicative of a position of the prosthetic limb 102 in space. The sensor data further includes pressure data obtained from pressure sensors 208 mounted in foot sole of the prosthetic limb 102. The pressure data is indicative force distribution across a foot and is used to derive weight bearing and balance parameters during standing or walking of the subject 100. The sensor data further includes electrical activity data obtained from electromyography (EMG) sensor 210 mounted in a thigh of the subject 100. The electrical activity data is indicative of electrical activity produced by skeletal muscles activity, which is indicative of subject intent for movement.

In an aspect, the sensor data includes at least one of: inclination angle data obtained from an inclinometer 212, which is indicative of a slope the subject 100 is navigating, and distance data obtained from an ultrasonic sensor 214, which is indicative of obstacle proximity. Both the inclination angle data and the distance data are used to derive a terrain type of the environment.

In an aspect, the predicted subject intent for movement includes at least one of starting, stopping or changing direction.

In an aspect, the data processing unit 216 is configured to filter the sensor data to remove noise and distortion to generate filtered sensor data. The data processing unit 216 is further configured to normalize the filtered sensor data to generate normalized sensor data having a consistent range or format across different sensors of the multimodal sensor array 202.

In an aspect, the data processing unit 216 is configured to extract features from the sensor data. The features are indicative of subject movement patterns and environmental conditions. The data processing unit 216 is further configured to execute the ML model 230 by providing the features as an input to obtain the predicted subject intent and the predicted prosthetic limb parameters.

In an aspect, the features include: features that are indicative of at least some of a walking speed of the subject 100, a terrain type of the environment, or an obstacle proximity in the environment.

In an aspect, the control signal is a combination of a first control signal generated by the fuzzy logic controller 232 and a second control signal generated by the PID controller 234. The fuzzy logic controller 232 is configured to: generate the first control signal for adjusting the prosthetic limb parameters based on a current position or movement of the prosthetic limb 102. The PID controller 234 is configured to: generate the second control signal to reduce any error between an actual position and a desired position of the prosthetic limb 102.

In an aspect, the fuzzy logic controller 232 includes: input membership functions 1400 that are configured to map angular movement data to specific gait cycle phases. The angular movement data is obtained using sensors mounted on an unaffected leg 110 of a specified subject 100. The fuzzy logic controller 232 further includes a rule-based fuzzy inference system that maps the specific gait cycle phases to corresponding prosthetic limb parameters for generating the control signal.

In an aspect, the PID controller 234 is configured to compensate for ground reaction forces during a gait cycle phase by providing additional torque adjustments to the prosthetic limb 102.

In an aspect, the data processing unit 216 is configured to determine slope data by obtaining inclination angle data and angular motion data for a specified period from the multimodal sensor array 202, comparing the inclination angle data with specified threshold angle ranges to classify a slope of a path into a first category of multiple categories, and determining adjustments to the prosthetic limb parameters based on the first category of the slope to generate adjusted prosthetic limb parameters.

In an aspect, the control system 218 is configured to generate the control signal for slope navigation based on the adjusted prosthetic limb parameters.

In an aspect, the data processing unit 216 is configured to determine obstacle avoidance by obtaining distance to potential obstacles in a path of the subject 100 from the multimodal sensor array 202, identifying a potential obstacle based on a specified minimum safe distance, determining an avoidance type and size of the potential obstacle, and determining adjustments to the prosthetic limb parameters based on the avoidance type and size of the potential obstacle to generate adjusted prosthetic limb parameters.

In an aspect, the control system 218 is configured to generate the control signal for obstacle avoidance based on the adjusted prosthetic limb parameters.

The second embodiment is illustrated with respect to FIG. 3-FIG. 17. The second embodiment discloses the process 300 for controlling the prosthetic limb 102 is described. The process 300 includes acquiring multimodal sensor data. The multimodal sensor data is collected from a plurality of sensors integrated into the prosthetic limb 102, including: accelerometers 204a-204b and gyroscopes 206 for linear and angular motion, pressure sensors 208 for force distribution across a foot, electromyography (EMG) sensors 210, and environmental sensors 226 including inclinometers 212 for slope detection and ultrasonic sensors 214 for obstacle detection. The process 300 further includes processing the sensor data. The processing includes. filtering and normalizing the sensor data to remove noise and ensure consistency in data format. The processing further includes extracting features from the sensor data to identify subject movement patterns and environmental conditions. The processing further includes generating predicted prosthetic limb parameters and predicted subject intent. The features are input into a machine learning (ML) model 230 trained on training data including sensor data and corresponding subject intent and corresponding prosthetic limb parameters. The ML model 230 outputting the predicted subject intent and the predicted prosthetic limb parameters. The processing further includes generating a control signal based on the processed sensor data. The control signal is generated by a control system 218 comprising a fuzzy logic controller 232 and a proportional-integral-derivative (PID) controller 234. The control signal is used to adjust the prosthetic limb parameters in real time.

In an aspect, the process 300 further includes sending the control signal to an actuator 222 of the prosthetic limb 102 for adjusting a movement of the prosthetic limb 102. The adjusting includes adjusting at least one of joint stiffness, damping, joint angles, motor torque, or motor speed.

In an aspect, controlling the prosthetic limb 102 includes: facilitating navigation of a slope or an incline of a path of a subject 100. Facilitating the navigation of the slope includes: obtaining inclination angle data and angular motion data for a specified period from the sensors, classifying the slope into a first category of multiple categories based on comparing the inclination angle data with specified threshold angle ranges, determining adjustments to the prosthetic limb parameters based on the first category of the slope to generate adjusted prosthetic limb parameters, and generating the control signal for slope navigation based on the adjusted prosthetic limb parameters.

In an aspect, controlling the prosthetic limb 102 includes: facilitating obstacle avoidance in a path of a subject 100. Facilitating the obstacle avoidance includes: acquiring distance data from ultrasonic sensors 214 to detect obstacles in a path of the subject 100, wherein the acquiring further includes filtering the distance data using a moving average filter to stabilize distance data and reduce noise, processing the distance data to derive obstacle parameters including a size, proximity, and a classification of an obstacle, determining adjustments to the prosthetic limb parameters based on the obstacle parameters to generate adjusted prosthetic limb parameters, and generating the control signal for obstacle avoidance based on the adjusted prosthetic limb parameters.

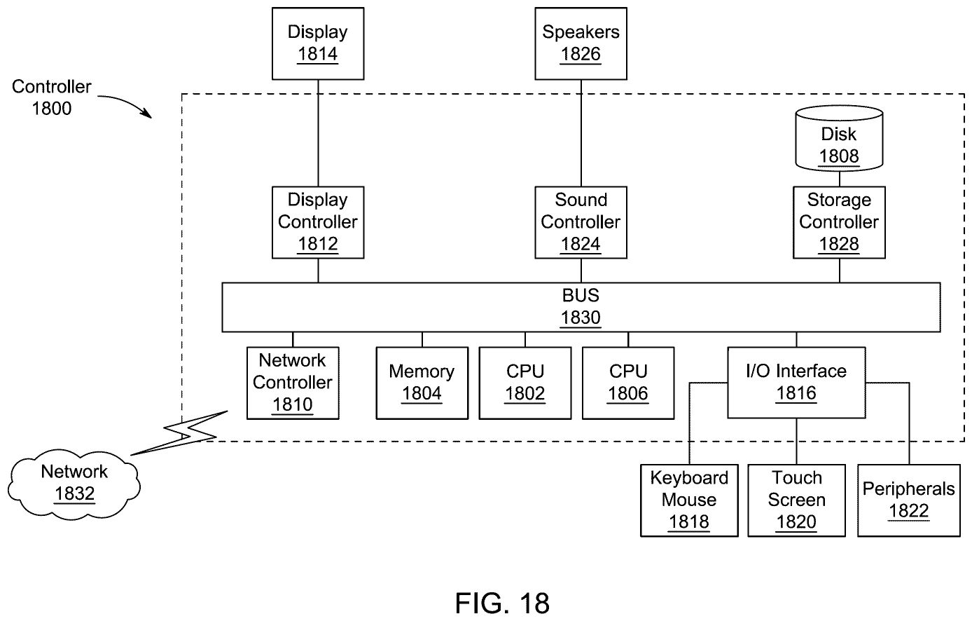

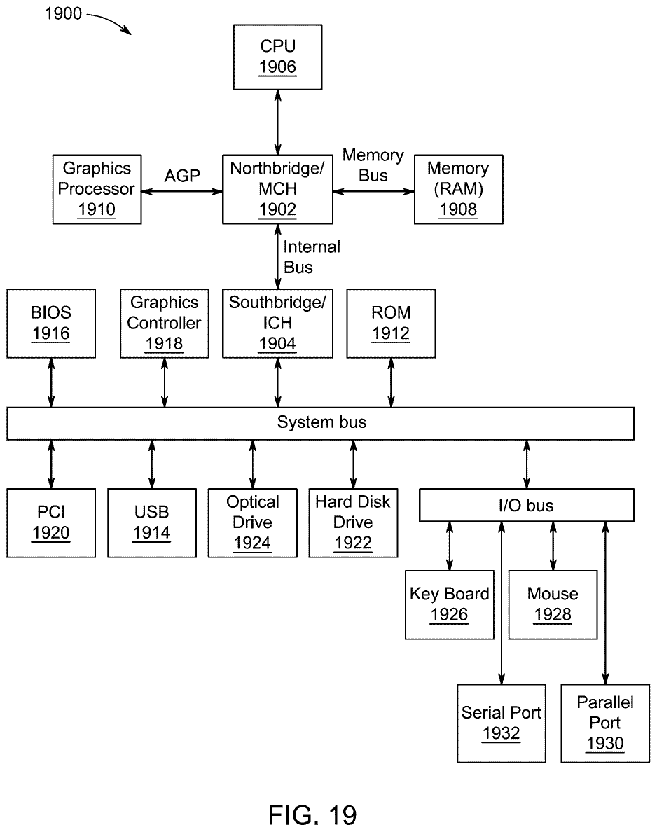

Next, further details of the hardware description of the computing environment according to exemplary embodiments are described with reference to FIG. 18. In FIG. 18, a controller 1800 is described as representative of the system 200 of FIG. 2A in which the controller 1800 includes a CPU 1802 which performs the processes described above/below. The process data and instructions may be stored in a memory 1804. These processes and instructions may also be stored on a storage medium disk 1808 such as a hard drive (HDD) or a portable storage medium or may be stored remotely.

Further, claims are not limited by the form of the computer-readable media on which the instructions of the inventive process are stored. For example, the instructions may be stored on compact discs (CDs), digital versatile disc (DVDs), in FLASH memory, read access memory (RAM), read only memory (ROM), programmable read only memory (PROM), erasable programmable read only memory (EPROM), electrically erasable programmable read only memory (EEPROM), hard disk or any other information processing device with which the computing device communicates, such as a server or computer.

Further, the claims may be provided as a utility application, background daemon, or component of an operating system, or combination thereof, executing in conjunction with CPU 1802, 1806 and an operating system such as Microsoft Windows 7, Microsoft Windows 10, UNiplexed Information Computing System (UNIX), Solaris, Lovable Intellect Not Using XP (LINUX), Apple Macintosh (MAC)-Operating System (OS) and other systems known to those skilled in the art.

The hardware elements in order to achieve the computing device may be realized by various circuitry elements, known to those skilled in the art. For example, CPU 1802 or CPU 1806 may be a Xenon or Core processor from Intel of America or an Opteron processor from advanced micro devices (AMD) of America, or may be other processor types that would be recognized by one of ordinary skill in the art. Alternatively, the CPU 1802, 1806 may be implemented on a field programmable Gate array (FPGA), application-specific integrated circuit (ASIC), programmable logic device (PLD) or using discrete logic circuits, as one of ordinary skill in the art would recognize. Further, CPU 1802, 1806 may be implemented as multiple processors cooperatively working in parallel to perform the instructions of the inventive processes described above.

The computing device in FIG. 18 also includes a network controller 1810, such as an Intel Ethernet PRO network interface card from Intel Corporation of America, for interfacing with network 1832. As can be appreciated, the network 1832 can be a public network, such as the Internet, or a private network such as a local area network (LAN) or a wide area network (WAN) network, or any combination thereof and can also include public switched telephone network, (PSTN) or an integrated services digital network (ISDN) sub-network. The network 1832 can also be wired, such as an Ethernet network, or can be wireless such as a cellular network including EDGE, 3G and 4G wireless cellular systems. The wireless network can also be Wireless Fidelity (WiFi), Bluetooth, or any other wireless form of communication that is known.

The computing device further includes a display controller 1812, such as a NVIDIA GeForce GTX or Quadro graphics adaptor from NVIDIA Corporation of America for interfacing with display 1814, such as a Hewlett Packard HPL2445w LCD monitor. A general purpose I/O interface 1816 interfaces with a keyboard and/or mouse 1818 as well as a touch screen panel 1820 on or separate from display 1814. General purpose I/O interface also connects to a variety of peripherals 1822 including printers and scanners, such as an OfficeJet or DeskJet from Hewlett Packard.

A sound controller 1824 is also provided in the computing device such as Sound Blaster X-Fi Titanium from Creative, to interface with speakers/microphone 1826 thereby providing sounds and/or music.

The general-purpose storage controller 1828 connects the storage medium disk 1808 with communication bus 1830, which may be an instruction set architecture (ISA), extended industry standard architecture (EISA), video electronics standards association (VESA), peripheral component interconnect (PCI), or similar, for interconnecting all of the components of the computing device. A description of the general features and functionality of the display 1814, keyboard and/or mouse 1818, as well as the display controller 1812, storage controller 1828, network controller 1810, sound controller 1824, and general purpose I/O interface 1816 is omitted herein for brevity as these features are known.

The exemplary circuit elements described in the context of the present disclosure may be replaced with other elements and structured differently than the examples provided herein. Moreover, circuitry configured to perform features described herein may be implemented in multiple circuit units (e.g., chips), or the features may be combined in circuitry on a single chipset, as shown on FIG. 19.

FIG. 19 is an exemplary schematic diagram of a data processing system 1900 used within the computing system, according to certain embodiments, for performing the functions of the exemplary embodiments. The data processing system 1900 is an example of a computer in which code or instructions implementing the processes of the illustrative embodiments may be located.

In FIG. 19, the data processing system 1900 employs a hub architecture including a north bridge and memory controller hub (NB/MCH) 1902 and a south bridge and input/output (I/O) controller hub (SB/ICH) 1904. The central processing unit (CPU) 1906 is connected to the NB/MCH 1902. The NB/MCH 1902 also connects to the memory 1908 via a memory bus, and connects to the graphics processor 1910 via an accelerated graphics port (AGP). The NB/MCH 1902 also connects to the SB/ICH 1904 via an internal bus (e.g., a unified media interface or a direct media interface). The CPU 1906 may contain one or more processors and even may be implemented using one or more heterogeneous processor systems.

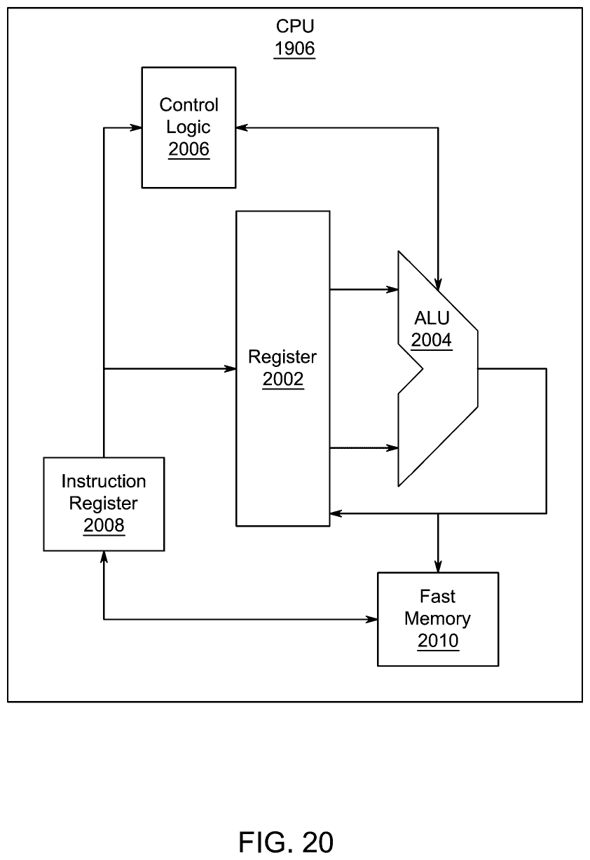

For example, FIG. 20 shows one implementation of the CPU 1906. In one implementation, the instruction register 2008 retrieves instructions from the fast memory 2010. At least part of these instructions is fetched from the instruction register 2008 by the control logic 2006 and interpreted according to the instruction set architecture of the CPU 1906. Part of the instructions can also be directed to the register 2002. In one implementation the instructions are decoded according to a hardwired method, and in another implementation the instructions are decoded according to a microprogram that translates instructions into sets of CPU configuration signals that are applied sequentially over multiple clock pulses. After fetching and decoding the instructions, the instructions are executed using the arithmetic logic unit (ALU) 2004 that loads values from the register 2002 and performs logical and mathematical operations on the loaded values according to the instructions. The results from these operations can be feedback into the register 2002 and/or stored in the fast memory 2010. According to certain implementations, the instruction set architecture of the CPU 1906 can use a reduced instruction set architecture, a complex instruction set architecture, a vector processor architecture, a very large instruction word architecture. Furthermore, the CPU 1906 can be based on a Von Neuman model or a Harvard model. The CPU 1906 can be a digital signal processor, the FPGA, the ASIC, the PLA, a PLD, or a CPLD. Further, the CPU 1906 can be an x86 processor by Intel or by AMD; an ARM processor, a Power architecture processor by, e.g., IBM; a SPARC architecture processor by Sun Microsystems or by Oracle; or other known CPU architecture.

Referring again to FIG. 19, the data processing system 1900 can include that the SB/ICH 1904 is coupled through a system bus to an I/O Bus, a read only memory (ROM) 1912, universal serial bus (USB) port 1914, a flash binary input/output system (BIOS) 1916, and a graphics controller 1918. PCI/PCIe devices can also be coupled to SB/ICH 1904 through a PCI bus 1920.

The PCI devices may include, for example, Ethernet adapters, add-in cards, and PC cards for notebook computers. The Hard disk drive 1922 and CD-ROM (optical drive) 1924 can use, for example, an integrated drive electronics (IDE) or serial advanced technology attachment (SATA) interface. In one implementation the I/O bus can include a super I/O (SIO) device.

Further, the hard disk drive (HDD) 1922 and optical drive 1924 can also be coupled to the SB/ICH 1904 through a system bus. In one implementation, a keyboard 1926, a mouse 1928, a parallel port 1930, and a serial port 1932 can be connected to the system bus through the I/O bus. Other peripherals and devices that can be connected to the SB/ICH 1904 using a mass storage controller such as SATA or PATA, an Ethernet port, an ISA bus, a LPC bridge, SMBus, a DMA controller, and an Audio Codec.

Moreover, the present disclosure is not limited to the specific circuit elements described herein, nor is the present disclosure limited to the specific sizing and classification of these elements. For example, the skilled artisan will appreciate that the circuitry described herein may be adapted based on changes on battery sizing and chemistry, or based on the requirements of the intended back-up load to be powered.

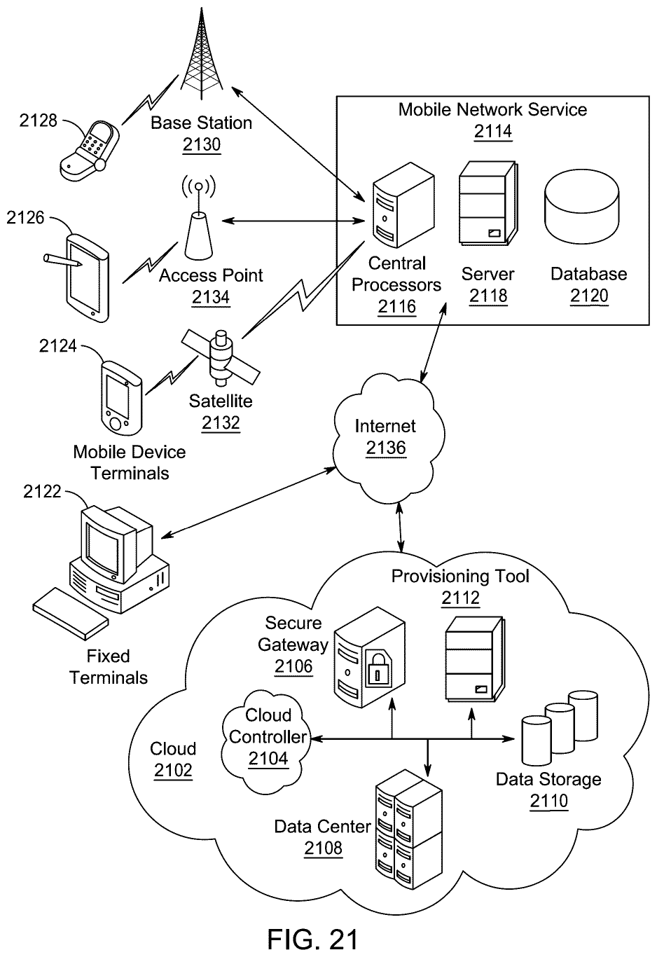

The functions and features described herein may also be executed by various distributed components of a system. For example, one or more processors may execute these system functions, wherein the processors are distributed across multiple components communicating in a network. The distributed components may include one or more client and server machines, such as cloud 2102 including a cloud controller 2104, a secure gateway 2106, a data center 2108, data storage 2110 and a provisioning tool 2112, and mobile network services 2114 including central processors 2116, a server 2118 and a database 2120, which may share processing, as shown by FIG. 21, in addition to various human interface and communication devices (e.g., display monitors 2122, smart phones 2124, tablets 2126, personal digital assistants (PDAs) 2128). The network may be a private network, such as a base station 2130, satellite 2132 or access point 2134, or be a public network, may such as the Internet 2136. Input to the system may be received via direct user input and received remotely either in real-time or as a batch process. Additionally, some implementations may be performed on modules or hardware that are not identical to those described. Accordingly, other implementations are within the scope that may be claimed.

The above-described hardware description is a non-limiting example of corresponding structure for performing the functionality described herein.

Numerous modifications and variations of the present disclosure are possible in light of the above teachings. It is therefore to be understood that the invention may be practiced otherwise than as specifically described herein.

1. A prosthetic limb for replacing a portion of a leg of a subject, comprising:
a prosthetic limb body, a prosthetic limb knee and a prosthetic limb ankle;
a multimodal sensor array embedded in the prosthetic limb body to obtain sensor data, wherein the sensor data are used to derive parameters that are indicative of (a) a physiological state of the subject and (b) an environment in a proximity of the prosthetic limb;
a data processing unit that is configured to process the sensor data to generate a processed sensor data, wherein the processed sensor data is generated using a machine learning (ML) model trained on training data including sensor data and corresponding subject intent and corresponding prosthetic limb parameters, the ML model outputting the processed sensor data as a predicted subject intent and predicted prosthetic limb parameters based on the sensor data; and
a control system that is configured to generate a control signal having control parameters for adjusting a movement of the prosthetic limb, wherein the control parameters include values of the prosthetic limb parameters to be adjusted, wherein the control system is configured to generate the control signal based on the processed sensor data and using a fuzzy logic controller and a proportional-integral-derivative (PID) controller of the control system.

2. The prosthetic limb of claim 1, further comprising:
an actuator that is configured to physically adjust the prosthetic limb by adjusting the prosthetic limb parameters based on the control parameters, wherein the prosthetic limb parameters include at least some of joint stiffness, joint angle, damping, motor speed or motor torque.

3. The prosthetic limb of claim 1, further comprising:
a haptic feedback system to provide haptic feedback regarding positioning and movement of the prosthetic limb.

4. The prosthetic limb of claim 1, wherein the sensor data includes at least one of:
accelerometer data obtained using accelerometers mounted near the knee and the ankle of the prosthetic limb, wherein the accelerometer data is used to derive a speed of the subject, a joint angle of joints of the prosthetic limb, both of which are indicative of a gait of the subject,
angular motion data obtained using gyroscopes mounted near the knee and ankle of the prosthetic limb, wherein the angular motion data is indicative of angular orientation of one or more joints of the prosthetic limb, which is indicative of a position of the prosthetic limb in space,
pressure data obtained from pressure sensors mounted in foot sole of the prosthetic limb, wherein the pressure data is indicative force distribution across a foot and is used to derive weight bearing and balance parameters during standing or walking of the subject, and
electrical activity data obtained from electromyography (EMG) sensor mounted in a thigh of the subject, wherein the electrical activity data is indicative of electrical activity produced by skeletal muscles activity, which is indicative of subject intent for movement.

5. The prosthetic limb of claim 1, wherein the sensor data includes at least one of:
inclination angle data obtained from an inclinometer, which is indicative of a slope the subject is navigating, and
distance data obtained from an ultrasonic sensor, which is indicative of obstacle proximity, wherein both the inclination angle data and the distance data are used to derive a terrain type of the environment.

6. The prosthetic limb of claim 1, wherein the predicted subject intent for movement includes at least one of starting, stopping or changing direction.

7. The prosthetic limb of claim 1, wherein the data processing unit is configured to:
filter the sensor data to remove noise and distortion to generate filtered sensor data, and
normalize the filtered sensor data to generate normalized sensor data having a consistent range or format across different sensors of the multimodal sensor array.

8. The prosthetic limb of claim 1, wherein the data processing unit is configured to:
extract features from the sensor data, wherein the features are indicative of subject movement patterns and environmental conditions, and
execute the ML model by providing the features as an input to obtain the predicted subject intent and the predicted prosthetic limb parameters.

9. The prosthetic limb of claim 8, wherein the features include:
features that are indicative of at least some of a walking speed of the subject, a terrain type of the environment, or an obstacle proximity in the environment.

10. The prosthetic limb of claim 1, wherein the control signal is a combination of a first control signal generated by the fuzzy logic controller and a second control signal generated by the PID controller, wherein:
the fuzzy logic controller is configured to:
generate the first control signal for adjusting the prosthetic limb parameters based on a current position or movement of the prosthetic limb, and
the PID controller is configured to:
generate the second control signal to reduce any error between an actual position and a desired position of the prosthetic limb.

11. The prosthetic limb of claim 10, wherein the fuzzy logic controller includes:
input membership functions that are configured to map angular movement data to specific gait cycle phases, wherein the angular movement data is obtained using sensors mounted on an unaffected leg of a specified subject, and
a rule-based fuzzy inference system that maps the specific gait cycle phases to corresponding prosthetic limb parameters for generating the control signal.

12. The prosthetic limb of claim 10, wherein the PID controller is configured to compensate for ground reaction forces during a gait cycle phase by providing additional torque adjustments to the prosthetic limb.

13. The prosthetic limb of claim 1, wherein the data processing unit is configured to determine slope data by:
obtaining inclination angle data and angular motion data for a specified period from the multimodal sensor array,
comparing the inclination angle data with specified threshold angle ranges to classify a slope of a path into a first category of multiple categories, and
determining adjustments to the prosthetic limb parameters based on the first category of the slope to generate adjusted prosthetic limb parameters.

14. The prosthetic limb of claim 13, wherein the control system is configured to generate the control signal for slope navigation based on the adjusted prosthetic limb parameters.

15. The prosthetic limb of claim 1, wherein the data processing unit is configured to determine obstacle avoidance by:
obtaining distance to potential obstacles in a path of the subject from the multimodal sensor array,
identifying a potential obstacle based on a specified minimum safe distance,
determining an avoidance type and size of the potential obstacle, and
determining adjustments to the prosthetic limb parameters based on the avoidance type and size of the potential obstacle to generate adjusted prosthetic limb parameters.

16. The prosthetic limb of claim 15, wherein the control system is configured to generate the control signal for obstacle avoidance based on the adjusted prosthetic limb parameters.

17. A method for controlling a prosthetic limb, the method comprising:
acquiring multimodal sensor data, wherein the multimodal sensor data is collected from a plurality of sensors integrated into the prosthetic limb, including: accelerometers and gyroscopes for linear and angular motion, pressure sensors for force distribution across a foot, electromyography (EMG) sensors, and environmental sensors including inclinometers for slope detection and ultrasonic sensors for obstacle detection;
processing the sensor data, wherein the processing includes:
filtering and normalizing the sensor data to remove noise and ensure consistency in data format,
extracting features from the sensor data to identify subject movement patterns and environmental conditions, and
generating predicted prosthetic limb parameters and predicted subject intent, wherein the features are input into a machine learning (ML) model trained on training data including sensor data and corresponding subject intent and corresponding prosthetic limb parameters, the ML model outputting the predicted subject intent and the predicted prosthetic limb parameters; and
generating a control signal based on the processed sensor data, wherein the control signal is generated by a control system comprising a fuzzy logic controller and a proportional-integral-derivative (PID) controller, wherein the control signal is used to adjust the prosthetic limb parameters in real time.

18. The method of claim 17, further comprising:
sending the control signal to an actuator of the prosthetic limb for adjusting a movement of the prosthetic limb, wherein the adjusting includes adjusting at least one of joint stiffness, damping, joint angles, motor torque, or motor speed.

19. The method of claim 17, wherein controlling the prosthetic limb includes:
facilitating navigation of a slope or an incline of a path of a subject, wherein facilitating the navigation of the slope includes:
obtaining inclination angle data and angular motion data for a specified period from the sensors,
classifying the slope into a first category of multiple categories based on comparing the inclination angle data with specified threshold angle ranges,
determining adjustments to the prosthetic limb parameters based on the first category of the slope to generate adjusted prosthetic limb parameters, and
generating the control signal for slope navigation based on the adjusted prosthetic limb parameters.

20. The method of claim 17, wherein controlling the prosthetic limb includes:
facilitating obstacle avoidance in a path of a subject, wherein facilitating the obstacle avoidance includes:
acquiring distance data from ultrasonic sensors to detect obstacles in a path of the subject, wherein the acquiring further includes filtering the distance data using a moving average filter to stabilize distance data and reduce noise,
processing the distance data to derive obstacle parameters including a size, proximity, and a classification of an obstacle,
determining adjustments to the prosthetic limb parameters based on the obstacle parameters to generate adjusted prosthetic limb parameters, and
generating the control signal for obstacle avoidance based on the adjusted prosthetic limb parameters.
